(19) United States
(12) Patent Application Publication
(10) Pub. No.: US 2026/0326017 A1
(43) Pub. Date: Oct. 22, 2026

(54) SYSTEMS FOR CARBON DIOXIDE CAPTURE USING FUNCTIONALIZED SORBENTS AND WATER MANAGEMENT

(71) Applicants: GE Vernova Infrastructure Technology LLC, Greenville, SC (US); GE VERNOVA TECHNOLOGY GMBH, Baden (CH)

(72) Inventors: Jie Jerry LIU, Schenectady, NY (US); Hongbo CAO, Schenectady, NY (US); Jinbo CAO, Schenectady, NY (US); Xiaolei SHI, Schenectady, NY (US); Yali SU, Schenectady, NY (US); Mark D. DOHERTY, Schenectady, NY (US); Anil R. DUGGAL, Schenectady, NY (US); David R. MOORE, Schenectady, NY (US)

(21) Appl. No.: 19/481,317

(22) PCT Filed: May 9, 2023
(86) PCT No.: PCT/US2023/021542

§ 371 Date: Nov. 4, 2025

(51) Int. Cl. B01D 53/04; B01J 20/22; B01J 20/32

(52) U.S. Cl. CPC B01D 53/0438; B01D 53/0454; B01J 20/226; B01J 20/3206; B01J 20/3219; B01J 20/3272; B01J 20/3293; B01D 2253/204; B01D 2257/504

(57) ABSTRACT

A capture system for use in capturing carbon dioxide, the capture system including an adsorbent bed including at least one adsorption module and a functionalized sorbent. The at least one adsorption module is oriented to receive a gas stream, adsorb carbon dioxide from the gas stream via the functionalized sorbent, and discharge an exhaust stream. The capture system further includes a contactor for use in dynamically controlling a temperature and a relative humidity of the at least one adsorption module and a controller configured to modulate the temperature and the relative humidity based on the functionalized sorbent to facilitate increasing an amount of carbon dioxide captured by the adsorbent bed.

### BACKGROUND OF THE INVENTION

The present disclosure relates generally to capture systems and, more specifically, to systems that facilitate optimizing the adsorption and desorption of carbon dioxide gas by an absorbent bed using water management and functionalized sorbents.

At least some known industrial and power generation processes may result in the production of a gas stream containing contaminants, such as in the form of carbon dioxide (CO₂). As such, capture systems may be used to facilitate removing the contaminants from the gas stream prior to an exhaust stream being released into the atmosphere. For example, carbon capture systems may be used to attempt to capture CO₂ and store it underground to reduce an amount of CO₂ released into the atmosphere.

At least some known carbon capture systems may use adsorbent beds to capture and release CO₂. In some known systems, to improve the adsorption capacity and efficiency of the system, solid sorbent materials may be used with adsorbent beds to enhance the adsorption and desorption of CO₂, as opposed to other known systems that use liquid-amine based CO₂ capture processes. One example of a solid sorbent material that has been used are chemi-sorbents, which adsorb CO₂ through reversible chemical reactions and the formation of ammonium carbamate, ammonium carbonate, and/or ammonium bicarbonate. Examples of chemi-sorbents include amine-functionalized silica particles and metal-organic frameworks (MOF). However, the effectiveness of chemi-sorbent systems is often limited by functionalization, and detailed operation conditions as well.

To facilitate increasing the amount of CO₂ captured, at least some known carbon capture systems use water to improve the efficiency of CO₂ capture. For example, carbon capture systems may use wet conditions to improve CO₂ adsorption performance. However, interference from the H₂O molecules may reduce the CO₂ adsorption capacity of the chemi-sorbent material used in the adsorbent beds if too much H₂O is adsorbed. Accordingly, there exists a need for capture systems that use functionalized chemi-sorbents in the presence of water to optimize the efficiency and productivity of carbon dioxide adsorption and desorption.

### BRIEF DESCRIPTION OF THE INVENTION

In one aspect, a capture system for use in capturing carbon dioxide is provided. The capture system includes an adsorbent bed comprising at least one adsorption module and a functionalized sorbent, the at least one adsorption module oriented to receive a gas stream, adsorb carbon dioxide from the gas stream via the functionalized sorbent, and discharge an exhaust stream. The capture system further includes a contactor for use in dynamically controlling a temperature and a relative humidity of the at least one adsorption module and a controller configured to modulate the temperature and the relative humidity based on the functionalized sorbent to facilitate increasing an amount of carbon dioxide captured by the adsorbent bed.

### BRIEF DESCRIPTION OF THE DRAWINGS

FIG. 1 is a schematic illustration of an exemplary capture system that may be used to capture CO₂.

FIG. 2 is a schematic illustration of an alternative capture system that may be used to capture CO₂.

FIG. 3 illustrates the exemplary types of moisture adsorption isotherms that may be possessed by the capture system of FIG. 2.

FIG. 4 is a schematic of an exemplary control system that may be used with the capture systems of FIG. 1 and FIG. 2.

FIG. 5 illustrates an exemplary potential H₂O adsorption capacity of sorbents functionalized with AEAM, spermidine, a first hybrid of amines (Hybrid Compound 1, spermine: AEAM=0.65:0.35), or a second hybrid of amines (Hybrid Compound 2, spermine: AEAM=0.32:0.46) measured at 25° C. in accordance with the present disclosure;

FIG. 6 illustrates an exemplary potential H₂O adsorption capacity of sorbents functionalized with the first hybrid of amines (Hybrid Compound 1, spermine: AEAM=0.65:0.35) as a function of relative humidity at varying temperatures in accordance with the present disclosure.

FIG. 7 illustrates an exemplary potential H₂O adsorption capacity of sorbents functionalized with the first hybrid of amines (Hybrid Compound 1, spermine: AEAM=0.65:0.35) as a function of H₂O partial pressure at varying temperatures in accordance with the present disclosure.

FIG. 8 illustrates an exemplary potential CO₂ and H₂O adsorption capacity of sorbents functionalized with the first hybrid of amines (Hybrid Compound 1, spermine: AEAM=0.65:0.35) as a function of relative humidity at 40° C. in accordance with the present disclosure.

FIG. 9 illustrates an exemplary potential CO₂ adsorption capacity of sorbents functionalized with the first hybrid of amines (Hybrid Compound 1, spermine: AEAM=0.65:0.35) as a function of water pressure at varying temperatures in accordance with the present disclosure.

FIG. 10 illustrates an exemplary potential H₂O adsorption capacity of sorbents functionalized with the first hybrid of amines (Hybrid Compound 1, spermine: AEAM=0.65:0.35) as a function of water pressure at varying temperatures in accordance with the present disclosure.

FIG. 11 illustrates an exemplary potential CO₂ adsorption capacity of sorbents functionalized with the first hybrid of amines (Hybrid Compound 1, spermine: AEAM=0.65:0.35) as a function of relative humidity at varying temperatures in accordance with the present disclosure.

FIG. 12 illustrates an exemplary potential CO₂ adsorption isotherms of sorbents functionalized with the first hybrid of amines (Hybrid Compound 1, spermine: AEAM=0.65:0.35) for dry CO₂ and wet CO₂ with 30% relative humidity at 40° C. in accordance with the present disclosure.

### DETAILED DESCRIPTION OF THE INVENTION

The embodiments described herein relate to systems that use functionalized chemi-sorbents in the presence of water to facilitate optimizing the adsorption and desorption of carbon dioxide by an adsorbent bed. The advantages of the systems described herein include, at least: (i) increasing the efficiency and performance of carbon dioxide adsorption and desorption due to the use of changes in temperature of the adsorbent bed; (ii) increasing the efficiency and performance of carbon dioxide adsorption and desorption due to the use of functionalized sorbents within the adsorbent bed; (iii) increasing the efficiency and performance of carbon dioxide adsorption and desorption due to the use of changes in water relative humidity within the adsorbent bed; and (iv) increasing the performance of the capture system due to the modulation of temperature and water relative humidity within one or more adsorption modules within the adsorbent bed based on the functionalized sorbent within the one or more adsorption modules.

When introducing elements of various embodiments disclosed herein, the articles “a,” “an,” “the,” and “said” are intended to mean that there are one or more of the elements. The terms “comprising,” “including,” and “having” are intended to be inclusive and mean that there may be additional elements other than the listed elements.

Unless otherwise indicated, approximating language, such as “generally,” “substantially,” and “about,” as used herein indicates that the term so modified may apply to only an approximate degree, as would be recognized by one of ordinary skill in the art, rather than to an absolute or perfect degree. Accordingly, a value modified by a term or terms such as “about,” “approximately,” and “substantially” is not to be limited to the precise value specified. In at least some instances, the approximating language may correspond to the precision of an instrument for measuring the value. Additionally, unless otherwise indicated, the terms “first,” “second,” etc. are used herein merely as labels, and are not intended to impose ordinal, positional, or hierarchical requirements on the items to which these terms refer. Moreover, reference to, for example, a “second” item does not require or preclude the existence of, for example, a “first” or lower-numbered item or a “third” or higher-numbered item.

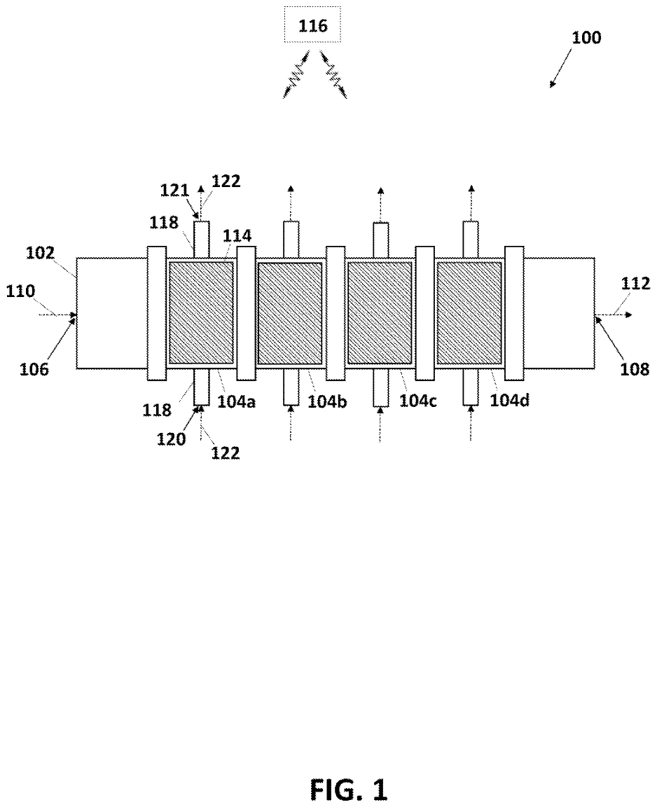

FIG. 1 is a schematic illustration of an exemplary capture system 100 that may be used to capture CO₂ using an adsorbent bed 102. In the exemplary embodiment, the absorbent bed 102 includes at least one adsorption module 104. More specifically, in the exemplary embodiment, the adsorbent bed 102 includes four adsorption modules 104a-d. In some embodiments, the capture system 100 may include more than or less than four adsorption modules 104. Moreover, in the exemplary embodiment, the adsorbent bed 102 includes an inlet 106 and an outlet 108. The inlet 106 and the outlet 108 are oriented such that during operation, a gas stream 110 received through the inlet 106 is channeled through each adsorption module 104 in series towards the outlet 108. As the gas stream 110 is channeled through each adsorption module 104, the adsorbent bed 102 captures CO₂ from the gas stream 110 and an exhaust stream 112 is discharged through the outlet 108.

The adsorption module 104 may include a solid sorbent to preferentially capture specific components of the gas stream 110, such as CO₂ and H₂O. In the exemplary embodiment, the adsorption module 104 includes a functionalized sorbent 114 including a sorbent and a functionalization ligand including at least one amine group such as, but not limited to, an aminosilicone group to facilitate enhancing the CO₂ capture capacity and productivity of the adsorbent bed 102. Generally, the functionalized sorbent 114 may be in any form known in the art that facilitates the systems described herein. For example, the functionalized sorbent 114 may be in the form of powder, composites mixed with binders, films or coatings, packed beds, and/or columns.

In the exemplary embodiment, after being received by the inlet 106, the gas stream 110 is channeled through a first module 104a of adsorption modules 104. Each of the absorption modules 104 includes the functionalized sorbent 114 to adsorb the CO₂ included in the gas stream 110. In some embodiments, the functionalized sorbent 114 may be the same for each adsorption module 104. In other embodiments, the functionalized sorbent 114 may vary within at least one adsorption module 104.

Generally, the gas stream 110 may be any suitable gas known in the art that facilitates the systems described herein. For example, the gas stream 110 may be air, flue gas, post-combustion gas, natural gas, and/or combinations thereof. In the exemplary embodiment, the gas stream 110 includes CO₂ and H₂O. In some embodiments, CO₂ may be present in the gas stream 110 in a range of from about 1 v % to about 10 v %. In other embodiments, CO₂ may be present in the gas stream 110 in an amount less than 1 v %, such as a volume of about or less than 300-500 ppm. In still other embodiments, CO₂ may be present in the gas stream 110 in an amount greater than 10 v %. In some embodiments, H₂O may be present in the gas stream 110 in a range of from about 0.01 v % to about 20 v %. In other embodiments, H₂O may be present in the gas stream 110 in an amount of less than 7 v %, such as approximately 2 v % to 7 v % as in the air during hot and humid summers, or less than 2 v % as in the air during cold winters. In still other embodiments, H₂O may be present in the gas stream 110 in an amount of more than 5 v %, such as in the post-combustion gases.

In the exemplary embodiment, the concentration of CO₂ of the gas stream 110 is generally highest as the gas stream 110 enters the inlet 106. As CO₂ is adsorbed by the functionalized sorbent 114 of each subsequent adsorption module 104, the concentration of CO₂ in the gas stream 110 is reduced as the gas stream 110 is channeled through the adsorption modules 104a-d towards the outlet 108. In the exemplary embodiment, the concentration of CO₂ in the gas stream 110 flowing through the adsorption modules 104a-d is lowest at the outlet 108 during a designated adsorption cycle.

In the exemplary embodiment, the capture system 100 also includes a controller 116 that dynamically adjusts operation of the capture system 100. For example, the controller 116 may facilitate maximizing the capture of CO₂ by changing the temperature of one or more adsorption module 104 and/or changing an H₂O content within one or more adsorption module 104, as described further herein.

The controller 116 modulates the operating conditions of the capture system 100 by dynamically adjusting the temperature of each adsorption module 104a-d. In the exemplary embodiment, the adsorption module 104 includes a contactor 118. The contactor 118 includes a contactor inlet 120 and a contactor outlet 121. In the exemplary embodiment, a first stream 122 received through the contactor inlet 118 modulates the temperature of the adsorption module 104 via heat transfer. The contactor 118 may use indirect or direct heat transfer to adjust the temperature of the adsorption module 104. For example, the contactor 118 may include a fluid circuit (not shown) defined between and extending from the contactor inlet 120 and to the contactor outlet 121, such that indirect heat transfer occurs between the first stream 122 flowing within the fluid circuit (not shown) and the functionalized sorbent 114 within the adsorption module 104. Additionally, for example, the contactor 118 may be in direct flow communication with the adsorption module 104, wherein direct heat transfer occurs between the first stream 122 and the functionalized sorbent 114 within the adsorption module 104. In some embodiments, the contactor 118 of one or more adsorption modules 104a-d may be coupled in series and/or in parallel.

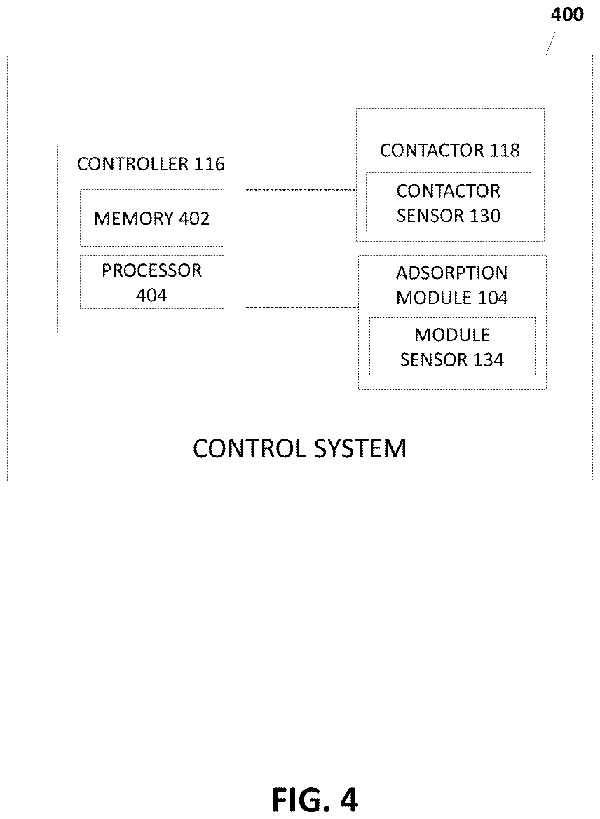

The controller 116 may adjust the temperature of one or more adsorption modules 104 while monitoring a regulated temperature T_{reg} of the first stream 122. In some embodiments, the first stream 122 may be in a liquid form. In other embodiments, the first stream 122 may be in a gaseous form. Heat transfer, either direct or indirect, between the first stream 122 and the functionalized sorbent 114 within one or more adsorption modules 104 facilitates controlling a temperature of the functionalized sorbent 114. The controller 116 may monitor the regulated temperature T_{reg} of the first stream 122 using a contactor sensor 130 (shown in FIG. 4). Additionally, the controller 116 may monitor a control temperature T_{cntl} of at least one adsorption module 104 using a module sensor 134 (shown in FIG. 4). In operating conditions where the control temperature T_{cntl} of at least one adsorption module 104 is lower than desired, the controller 124 may selectively increase the regulated temperature T_{reg} of the first stream 122, thereby increasing the temperature of the at least one adsorption module 104. Alternatively, in operating conditions where the control temperature T_{cntl} of at least one adsorption module 104 is higher than desired, the controller 124 may selectively decrease the regulated temperature T_{reg} of the first stream 122, thereby reducing the temperature of the at least one adsorption module 104.

Generally, the regulated temperature T_{reg} of the first stream 122, and thereby the temperature of the adsorption module 104, may be any suitable temperature known in the art that facilitates the capture of CO₂ by the systems described herein. In some embodiments, the regulated temperature T_{reg} of the first stream 122 may be in a range of from about 0° C. to about 150° C. In other embodiments, the regulated temperature T_{reg} of the first stream 122 may be in a range of from about 60° C. to about 250° C. In the exemplary embodiment, the regulated temperature T_{reg} of the first stream 122 is monitored within each adsorption module 104a-d. In some embodiments, the regulated temperature T_{reg} of the first stream 122 may be substantially uniform across each adsorption module 104. In other embodiments, the regulated temperature T_{reg} of the first stream 122 may vary across different adsorption modules 104a-d.

Additionally, the controller 116 may vary the regulated temperature T_{reg} of the first stream 122 within any of the adsorption modules 104a-d. For example, one or more adsorption modules 104a-d may include more than one module sensor 134 (shown in FIG. 4). Thus, the controller 116 may create a temperature profile that includes varying values of the regulated temperature T_{reg} of the first stream 122 within any or all of the adsorption modules 104a-d. In some embodiments, the varying values of the regulated temperature T_{reg} of the first stream 122 may form a gradient temperature profile within any or all of the adsorption modules 104a-d. In other embodiments, the varying values of the regulated temperature T_{reg} of the first stream 122 may form a discrete temperature profile within any or all of the adsorption modules 104a-d.

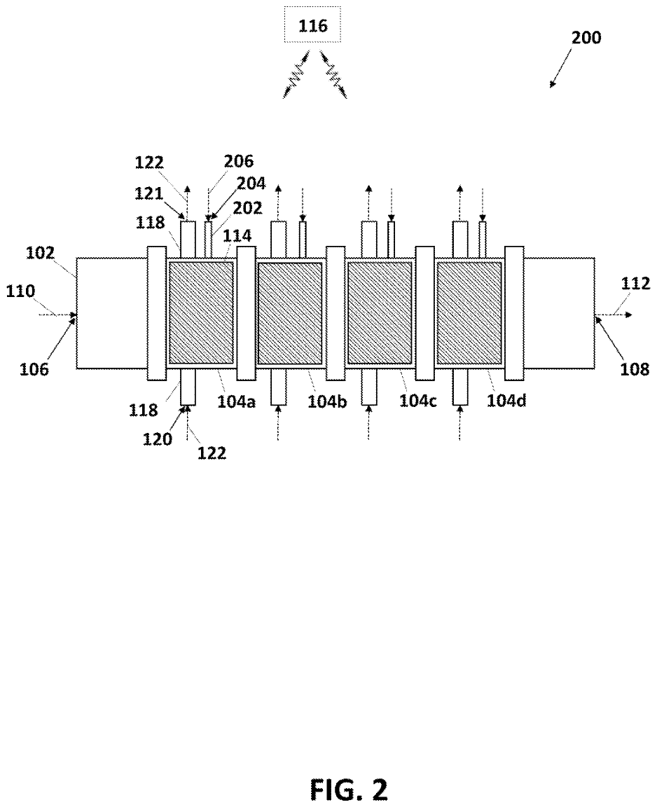

FIG. 2 is a schematic illustration of an exemplary capture system 200 that may be used to capture CO₂ using the adsorbent bed 102. The embodiment illustrated in FIG. 2 is similar to the embodiment illustrated in FIG. 1, with the differences noted herein, and as such, the same reference numbers are used in FIG. 2 as were used in FIG. 1. The controller 116 facilitates modulating the operating conditions of the capture system 200 by dynamically adjusting an H₂O relative humidity through adjusting the H₂O content within each adsorption module 104a-d. In the exemplary embodiment, the adsorption module 104 includes an injector 202 including an injector inlet 204. A second stream 206 received through the injector inlet 204 facilitates modulating the H₂O relative humidity by adjusting H₂O content within the adsorption module 104. In the exemplary embodiment, the second stream 206 includes H₂O. The second stream 206 may include H₂O in a liquid (e.g., water) or a gas (e.g., water vapor) form. In some embodiments, H₂O may be present in the second stream 206 in a range of from about 0.1 v % to about 20 v %. In other embodiments, H₂O may be present in the second stream 206 in a range of from about 4 v % to about 15 v %. In still other embodiments, H₂O may be present in the second stream 206 in a range of from about 0.5 v % to about 4 v %, such as generally occurs in ambient air.

The controller 116 may adjust the H₂O content within one or more adsorption modules 104 by regulating the flow of the second stream 206 into one or more adsorption modules 104. For example, the controller 116 may monitor relative humidity level for an H₂O concentration C of the adsorption module 104 using the module sensor 134 (shown in FIG. 4). In operating conditions where the H₂O relative humidity level for the H₂O concentration C is lower than desired in at least one adsorption module 104, the controller 116 may selectively increase the flow of the second stream 206, thereby injecting additional H₂O into the at least one adsorption module 104. In operating conditions where the H₂O relative humidity level is higher than desired in at least one adsorption module 104, the controller 116 may selectively decrease the flow of the second stream 206, thereby reducing the amount of additional H₂O injected into the at least one adsorption module 104. In another embodiment, at the beginning of the adsorption cycle, the controller 116 may selectively increase the flow of the second stream 206, thereby injecting additional H₂O into the at least one adsorption module 104 to increase the relative humidity beyond the desirable relative humidity level to allow fast H₂O adsorption. Then at later stage of the adsorption cycle, the controller 116 may selectively decrease or stop the flow of the second stream 206 to reduce relative humidity level of the at least one adsorption module 104.

The controller 116 may also adjust the H₂O relative humidity within one or more adsorption modules 104 by monitoring the regulated temperature T_{reg} of the first stream 122 of one or more absorption modules 104. In operating conditions where the H₂O relative humidity level is higher than desired in at least one adsorption module 104, the controller 116 may selectively increase the regulated temperature T_{reg} of the first stream 122, thereby increasing the temperature of at least one adsorption module 104, causing a decrease in the H₂O relative humidity due to increased saturated vapor pressure at elevated temperatures. In operating conditions where the H₂O relative humidity is lower than desired in at least one adsorption module 104, the controller 116 may selectively decrease the regulated temperature T_{reg} of the first stream 122, thereby decreasing the temperature of at least one adsorption module 104 and causing an increase in the H₂O relative humidity. The regulated temperature T_{reg} of the first stream 122 may be increased or decreased using any systems described herein.

The controller 116 modulates the operating conditions of the capture system 200 to facilitate optimizing the adsorption and desorption of CO₂ by maintaining a relative humidity of each adsorption module 104a-d. The relative humidity of each adsorption module 104a-d may be based on the regulated temperature T_{reg} of the first stream 122 and on the H₂O concentration C of the adsorption module 104. In the exemplary embodiment, the controller 116 adjusts the regulated temperature T_{reg} of the first stream 122 and the H₂O concentration C of the adsorption module 104 based on its H₂O adsorption isotherm of the functionalized sorbent 114 within the adsorption module 104 to facilitate optimizing the adsorption and desorption of CO₂ through monolayer adsorption of H₂O.

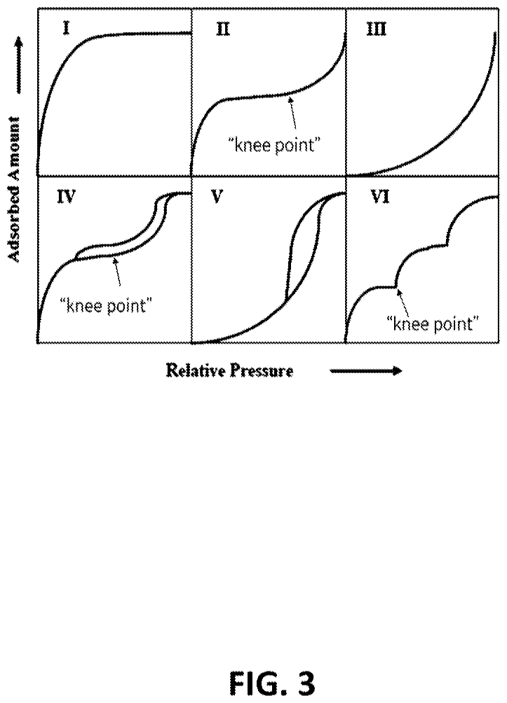

Generally, the capture system 200 may possess any suitable adsorption isotherm that facilitates the capture of CO₂ by the systems described herein. There are six types of moisture adsorption isotherms as defined by IUPAC, the shapes of which are dependent on relative humidity and temperature (shown in FIG. 3). Of these six types, type II, type IV, and type VI each display a flattened region (e.g. an “inflection point”) up to a knee point generally corresponding to monolayer coverage and adsorption of H₂O (shown in FIG. 3). In the exemplary embodiment, the controller 116 modulates the operating conditions of the capture system 200 for monolayer coverage and adsorption of H₂O by the functionalized sorbent 114, thereby facilitating optimizing the adsorption and desorption of CO₂. For example, the capture system 200 may possess a type II, type IV, or type VI adsorption isotherm (shown in FIG. 3), which are particularly advantageous to ensure monolayer coverage and adsorption of H₂O, thereby increasing the CO₂ adsorption capacity of the functionalized sorbent 114.

In addition to being dependent on relative humidity and temperature, adsorption isotherms are dependent on the sorbent material. Generally, the functionalized sorbent 114 may be any functionalized sorbent material that facilitates the capture of CO₂ by the systems described herein. In the exemplary embodiment, the functionalized sorbent 114 includes a sorbent and at least one functionalization ligand that includes an aminosilicone group to optimize the capacity and productivity of CO₂ capture relative to the adsorption of H₂O.

In some embodiments, the functionalized sorbent includes a first type of functionalization ligand, wherein the first type of functionalization ligand includes at least one functionalization ligand that includes an aminosilicone group. Generally, the at least one functionalization ligand that includes an aminosilicone group may include any such suitable ligand that facilitates the functionalized sorbent described herein. The at least one functionalization ligand that includes an aminosilicone group may include only one functionalization ligand that includes an aminosilicone group or two or more functionalization ligands that each include an aminosilicone group.

Generally, the sorbent may be any suitable sorbent known in the art that facilitates the functionalized sorbent described herein. In some embodiments, the sorbent is selected from the group consisting of coordination framework compounds, metal-organic framework (MOF) compounds, porous coordination polymers (PCPs), covalent organic framework (COF) compounds, zeolitic imidazolate framework (ZIF) compounds, crystalline porous materials, crystalline open frameworks, reticular chemistry, silica particles, zeolites, silico-alumino-phosphates (SAPOs), alumino-phosphates (AlPOs), polyaromatic frameworks (PAFs), activated carbons, molecular organic solids, and combinations thereof.

As used herein, MOF compounds are a class of compounds including metal ions or clusters coordinated to organic ligands to form one-, two-, or three-dimensional structures. The metal ions or clusters act as joints and are bound by multidirectional organic ligands, which act as linkers in a network structure. MOF compounds have a modular nature that allows for synthetic tunability, which affords fine chemical and structural control. Properties such as porosity, stability, particle morphology, and conductivity can be tailored for specific applications.

In many embodiments, the sorbent is a MOF compound including a MOF metal or metal-containing cluster and a MOF linker.

In some embodiments, the MOF metal may be any suitable MOF metal known in the art that facilitates the functionalized sorbent described herein. In other embodiments, the MOF metal is a metal selected from the group consisting of alkali metals, alkaline earth metals, transition metals, Ca, Mn, Cr, Fe, Co, Ni, Cu, Zn, ions thereof, hydrates thereof, salts thereof, halides thereof, fluorides thereof, chlorides thereof, bromides thereof, iodides thereof, nitrates thereof, acetates thereof, sulfates thereof, phosphates thereof, carbonates thereof, oxides thereof, formates thereof, carboxylates thereof, and combinations thereof. In some embodiments, the MOF metal includes Mg.

In some embodiments, the MOF metal-containing cluster may be any suitable MOF metal-containing cluster known in the art that facilitates the functionalized sorbent described herein. In some embodiments, the MOF metal-containing cluster includes an MOF metal node and a linker strut, with the MOF metal and the linker each defined as described herein. In other embodiments, the MOF metal-containing cluster includes an MOF metal-oxy cluster.

In some embodiments, the MOF linker may be any suitable MOF linker known in the art that facilitates the functionalized sorbent described herein. Generally, the geometry and connectivity of a linker contribute to the structure of the resulting MOF compound. Adjustments of linker geometry, length, ratio, and functional-group can tune the size, shape, and internal surface property of a MOF compound for a targeted application.

In at least some embodiments, the MOF linker is a linker selected from the group consisting of polytopic linkers, ditopic linkers, tritopic linkers, tetratopic linkers, pentatopic linkers, hexatopic linkers, heptatopic linkers, octatopic linkers, mixed linkers, desymmetrized linker, metallo linkers, N-heterocyclic linkers, and combinations thereof.

In at least some embodiments, the MOF linker is a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (H₄TDA), 4,4′-Dihydroxyazobenzene-3,3′-dicarboxylic acid (H₄OSA), protonated, partially and fully deprotonated forms thereof, and combinations thereof. As another example, in at least some embodiments, the MOF linker is a linker selected from the group consisting of dicarboxylates (e.g., terephthalic acid), tricarboxylates (e.g., 1,3,5-benzentricarboxylic acid), azolates, tetrazolates, and combinations thereof.

As another example, in at least some embodiments, the MOF linker is a dicarboxylic acid linker selected from the group consisting of 1,4-butanedicarboxylic acid, 4-oxopyran-2,6-dicarboxylic acid, 1,6-hexanedicarboxylic acid, decanedicarboxylic acid, 1,8-heptadecanedicarboxylic acid, 1,9-heptadecanedicarboxylic acid, heptadecanedicarboxylic acid, acetylenedicarboxylic acid, 1,2-benzenedicarboxylic acid, 2,3-pyridinedicarboxylic acid, pyridine-2,3-dicarboxylic acid, 1,3-butadiene-1,4-dicarboxylic acid, 1,4-benzenedicarboxylic acid, p-benzenedicarboxylic acid, imidazole-2,4-dicarboxylic acid, 2-methylquinoline-3,4-dicarboxylic acid, quinoline-2,4-dicarboxylic acid, quinoxaline-2,3-dicarboxylic acid, 6-chloroquinoxaline-2,3-dicarboxylic acid, 4,4′-diaminophenylmethane-3,3′-dicarboxylic acid, quinoline-3,4-dicarboxylic acid, 7-chloro-4-hydroxyquinoline-2,8-dicarboxylic acid, diimidedicarboxylic acid, pyridine-2,6-dicarboxylic acid, 2-methylimidazole-4,5-dicarboxylic acid, thiophene-3,4-dicarboxylic acid, 2-isopropylimidazole-4,5-dicarboxylic acid, tetrahydropyran-4,4-dicarboxylic acid, perylene-3,9-dicarboxylic acid, perylenedicarboxylic acid, Pluriol E 200-dicarboxylic acid, 3,6-dioxaoctanedicarboxylic acid, 3,5-cyclohexadiene-1,2-dicarboxylic acid, octanedicarboxylic acid, pentane-3,3-carboxylic acid, 4,4′-diamino-1,1′-diphenyl-3,3′-dicarboxylic acid, 4,4′-diaminodiphenyl-3,3′-dicarboxylic acid, benzidine-3,3′-dicarboxylic acid, 1,4-bis-(phenylamino)benzene-2,5-dicarboxylic acid, 1,1′-dinaphthyl-8,8′-dicarboxylic acid, 7-chloro-8-methylquinoline-2,3-dicarboxylic acid, 1-anilinoanthraquinone-2,4′-dicarboxylic acid, polytetrahydrofuran-250-dicarboxylic acid, 1,4-bis(carboxymethyl) piperazine-2,3-dicarboxylic acid, 7-chloroquinoline-3,8-dicarboxylic acid, 1-(4-carboxy)phenyl-3-(4-chloro) phenylpyrazoline-4,5-dicarboxylic acid, 1,4,5,6,7,7,-hexachloro-5-norbornene-2,3-dicarboxylic acid, phenylindanedicarboxylic acid, 1,3-dibenzyl-2-oxoimidazolidine-4,5-dicarboxylic acid, 1,4-cyclohexanedicarboxylic acid, naphthalene-1,8-dicarboxylic acid, 2-benzoylbenzene-1,3-dicarboxylic acid, 1,3-dibenzyl-2-oxoimidazolidine-4,5-cisdicarboxylic acid, 2,2′-biquinoline-4,4′-dicarboxylic acid, pyridine-3,4-dicarboxylic acid, 3,6,9-trioxaundecanedicarboxylic acid, o-hydroxybenzophenonedicarboxylic acid, Pluriol E 300-dicarboxylic acid, Pluriol E 400-dicarboxylic acid, Pluriol E 600-dicarboxylic acid, pyrazole-3,4-dicarboxylic acid, 2,3-pyrazinedicarboxylic acid, 5,6-dimethyl-2,3-pyrazinedicarboxylic acid, 4,4′-diaminodiphenyletherdiimidedicarboxylic acid, 4,4′-diaminodiphenylmethanediimidedicarboxylic acid, 4,4′-diaminodiphenylsulfonediimidedicarboxylic acid, 2,6-naphthalenedicarboxylic acid, 1,3-adamantanedicarboxylic acid, 1,8-naphthalenedicarboxylic acid, 2,3 naphthalenedicarboxylic acid, 8-methoxy-2,3-naphthalenedicarboxylic acid, 8-nitro-2,3-naphthalenedicarboxylic acid, 8-sulfo-2,3-naphthalenedicarboxylic acid, anthracene-2,3-dicarboxylic acid, 2′-3′-diphenyl-p-terphenyl-4,4″-dicarboxylic acid, diphenylether-4,4′-dicarboxylic acid, imidazole-4,5-dicarboxylic acid, 4 (1H)-oxothiochromene-2,8-dicarboxylic acid, 5-t-butyl-1,3-benzenedicarboxylic acid, 7,8-quinolinedicarboxylic acid, 4,5-imidazoledicarboxylic acid, 4-cyclohexene-1,2-dicarboxylic acid, hexatriacontanedicarboxylic acid, tetradecanedicarboxylic acid, 1,7-heptanedicarboxylic acid, 5-hydroxy-1,3-benzenedicarboxylic acid, pyrazine-2,3-dicarboxylic acid, furan-2,5-dicarboxylic acid, 1-nonene-6,9-dicarboxylic acid, eicosenedicarboxylic acid, 4,4′-dihydroxydiphenylmethane-3,3′-dicarboxylic acid, 1-amino-4-methyl-9,10-dioxo-9,10-dihydroanthracene-2,3-dicarboxylic acid, 2,5-pyridinedicarboxylic acid, cyclohexene-2,3-dicarboxylic acid, 2,9-dichlorofluorubin-4,11-dicarboxylic acid, 7-chloro-3-methylquinoline-6,8-dicarboxylic acid, 2,4-dichlorobenzophenone-2′,5′-dicarboxylic acid, 1,3-benzenedicarboxylic: acid, 1-methylpyrrole-3,4-dicarboxylic acid, 1-benzyl-1H-pyrrole-3,4-dicarboxylic acid, anthraquinone-1,5-dicarboxylic acid, 3,5-pyrazoledicarboxylic acid, 2-nitrobenzene-1,4-dicarboxylic acid, heptane-1,7-dicarboxylic acid, cyclobutane-1,1-dicarboxylic acid, 1,14-tetradecanedicarboxylic acid, 5,6-dehydronorbornane-2,3-dicarboxylic acid, 5-ethyl-2,3-pyridinedicarboxylic acid, and combinations thereof.

As another example, in at least some embodiments, the MOF linker is a tricarboxylic acid linker selected from the group consisting of 2-hydroxy-1,2,3-propanetricarboxylic acid, 7-chloro-2,3,8-quinolinetricarboxylic acid, 1,2,4-benzenetricarboxylic acid, 1,2,4-butanetricarboxylic acid, 2-phosphono-1,2,4-butanetricarboxylic acid, 1,3,5-benzenetricarboxylic acid, 1-hydroxy-1,2,3-propanetricarboxylic acid, 4,5-dihydro-4,5-dioxo-1H-pyrrolo[2,3-F]quinoline-2,7,9-tricarboxylic acid, 5-acetyl-3-amino-6-methylbenzene-1,2,4-tricarboxylic acid, 3-amino-5-benzoyl-6-methylbenzene-1,2,4-tricarboxylic acid, 1,2,3-propanetricarboxylic acid, aurinetricarboxylic acid, and combinations thereof.

As another example, in at least some embodiments, the MOF linker is a tetracarboxylic acid linker selected from the group consisting of 1,1-dioxide-perylo[1,12-BCD]thiophene-3,4,9,10-tetracarboxylic acid, perylenetetracarboxylic acids, perylene-3,4,9,10-tetracarboxylic acid, perylene-1,12-sulfone-3,4,9,10-tetracarboxylic acid, butanetetracarboxylic acids, 1,2,3,4-butanetetracarboxylic acid, meso-1,2,3,4-butanetetracarboxylic acid, decane-2,4,6,8-tetracarboxylic acid, 1,4,7,10,13,16-hexaoxacyclooctadecane-2,3,11,12-tetracarboxylic acid, 1,2,4,5-benzenetetracarboxylic acid, 1,2,11,12-dodecanetetracarboxylic acid, 1,2,5,6-hexanetetracarboxylic acid, 1,2,7,8-octanetetracarboxylic acid, 1,4,5,8-naphthalenetetracarboxylic acid, 1,2,9,10-decanetetracarboxylic acid, benzophenonetetracarboxylic acid, 3,3′,4,4′-benzophenonetetracarboxylic acid, tetrahydrofurantetracarboxylic acid, cyclopentanetetracarboxylic acids, cyclopentane-1,2,3,4-tetracarboxylic acid, and combinations thereof.

In the exemplary embodiment, the MOF linker is 4,4′-dihydroxy-[1,1′-biphenyl]-3,3′-dicarboxylic acid (H₄dobpdc) and/or 4,4′-dioxidobiphenyl-3,3′-dicarboxylate (dobpdc⁴⁻). In some embodiments, dobpdc includes 4,4′-dihydroxy-[1,1′-biphenyl]-3,3′-dicarboxylic acid, its mono-carboxylate form, its di-carboxylate form, its mono-phenoxide form, its di-phenoxide form, and combinations thereof.

In some embodiments, the MOF linker is one or more of the following linkers:

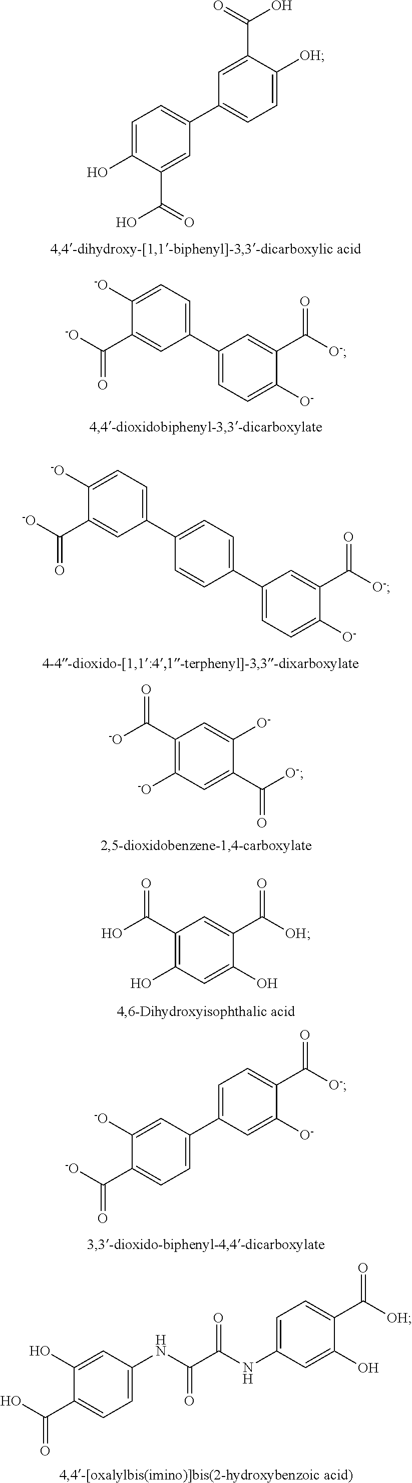

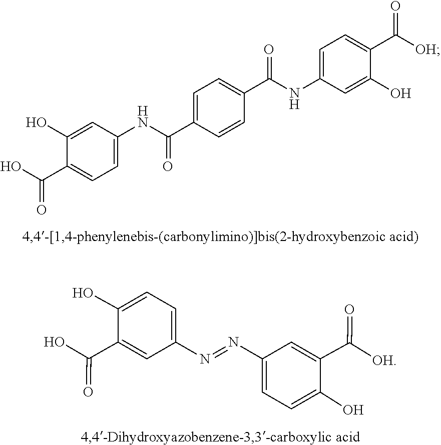 [SMILES: O=C(Nc1ccc(C(=O)O)c(O)c1)c1ccc(C(=O)Nc2ccc(C(=O)O)c(O)c2)cc1 ; O=C(O)c1cc(/N=N/c2ccc(O)c(C(=O)O)c2)ccc1O]

In some embodiments, the MOF compound is a MOF compound of the MOF-74 family. In some embodiments, the MOF compound is a MOF compound of the MOF-303 family. In some embodiments, the MOF compound is Mg₂(dobpdc).

In some embodiments, the functionalized sorbent is a functionalized MOF compound of Formula (I)

$${M}_{x} {L}_{y} {F}_{a}^{A} {F}_{b}^{B} , \qquad ( Formula I )$$

wherein:
M is a MOF metal or metal-containing cluster;
L is a MOF linker;
F^{A} is at least one functionalization ligand comprising an aminosilicone group;
F^{B} is at least one functionalization ligand not comprising an aminosilicone group;
x is a value in a range of 1 to 6;
y is a value in a range of 1 to 6;
a is a value greater than 0 and less than or equal to 2; and
b is a value in a range of 0 to 2.

In some embodiments, the functionalized sorbent includes a second type of functionalization ligand, wherein the second type of functionalization ligand includes at least one functionalization ligand that does not include an aminosilicone group. In some embodiments, the functionalized sorbent also includes at least one functionalization ligand that does not include an aminosilicone group. Generally, the at least one functionalization ligand that does not include an aminosilicone group may include any such suitable ligand that facilitates the functionalized sorbent described herein. The at least one functionalization ligand that does not include an aminosilicone group may include only one functionalization ligand that does not include an aminosilicone group or two or more functionalization ligands that each do not include an aminosilicone group.

In some embodiments, the at least one functionalization ligand that does not include an aminosilicone group is selected from the group consisting of amine ligands, monoamine ligands, diamine ligands, triamine ligands, tetra-amine ligands, penta-amine ligands, hexa-amine ligands, polyamine ligands, alkylamine ligands, and amino-alcohol ligands. Exemplary ligands include, but are not limited to, ethylene diamine, N-methylethylenediamine, N-ethylethylenediamine, N,N-dimethylethylenediamine, N,N-diethylethylenediamine, di(N-methyl)ethylene diamine, N-isopropylethylenediamine, N,N-dimethyl-N-methylethylene diamine, di(N,N-dimethyl)ethylene di amine, N,N-diisopropylethylene diamine, 2,2-dimethyl-1,3-diaminopropane, 1,3-diaminopentane, diethylenetriamine, N-(2-aminoethyl)-1,3-propanediamine, bis(3-aminopropyl)amine, N-(3-aminopropyl)-1,4-diaminobutane (spermidine), triethylenetetramine, N,N′-bis(2-aminoethyl)-1,3-propanediamine, 1,2-bis(3-aminopropylamino) ethane, N,N′-bis(3-aminopropyl)-1,3-propanediamine, N,N′-bis(3-aminopropyl)-1,4-diaminobutane (spermine), tetraethylenepentamine, and/or combinations thereof.

Generally, the at least one functionalization ligand including an aminosilicone group and the at least one functionalization ligand not including an aminosilicone group may be present in any suitable ratio known in the art that facilitates the functionalized sorbent described herein. In some embodiments, the ratio is selected from the group consisting of a molar ratio, a weight ratio, and a volume ratio. In some embodiments, the at least one functionalization ligand including an aminosilicone group and the at least one functionalization ligand not including an aminosilicone group are present in a ratio in a range of from about 10:1 to about 1:10. In some embodiments, the at least one functionalization ligand including an aminosilicone group and the at least one functionalization ligand not including an aminosilicone group are present in a ratio in a range of from about 9:1 to about 1:9. In some embodiments, the at least one functionalization ligand including an aminosilicone group and the at least one functionalization ligand not including an aminosilicone group are present in a ratio in a range of from about 8:1 to about 1:8. In some embodiments, the at least one functionalization ligand including an aminosilicone group and the at least one functionalization ligand not including an aminosilicone group are present in a ratio in a range of from about 7:1 to about 1:7. In some embodiments, the at least one functionalization ligand including an aminosilicone group and the at least one functionalization ligand not including an aminosilicone group are present in a ratio in a range of from about 6:1 to about 1:6. In some embodiments, the at least one functionalization ligand including an aminosilicone group and the at least one functionalization ligand not including an aminosilicone group are present in a ratio in a range of from about 5:1 to about 1:5. In some embodiments, the at least one functionalization ligand including an aminosilicone group and the at least one functionalization ligand not including an aminosilicone group are present in a ratio in a range of from about 4:1 to about 1:4. In some embodiments, the at least one functionalization ligand including an aminosilicone group and the at least one functionalization ligand not including an aminosilicone group are present in a ratio in a range of from about 3:1 to about 1:3. In some embodiments, the at least one functionalization ligand including an aminosilicone group and the at least one functionalization ligand not including an aminosilicone group are present in a ratio in a range of from about 2:1 to about 1:2. In some embodiments, the at least one functionalization ligand including an aminosilicone group and the at least one functionalization ligand not including an aminosilicone group are present in a ratio of about 1:1.

In some embodiments, the at least one functionalization ligand including an aminosilicone group is present in a lesser amount than the at least one functionalization ligand not including an aminosilicone group.

In some embodiments, the at least one functionalization ligand including an aminosilicone group and the at least one functionalization ligand not including an aminosilicone group are present in a ratio of about 1:2, about 1:3, about 1:4, about 1:5, about 1:6, about 1:7, about 1:8, about 1:9, or about 1:10.

In many embodiments, the at least one functionalization ligand including an aminosilicone group may be any suitable at least one functionalization ligand including an aminosilicone group known in the art that facilitates the functionalized sorbent described herein.

In some embodiments, the at least one functionalization ligand including an aminosilicone group includes at least one amine selected from the group consisting of primary amines, secondary amines, tertiary amines, and combinations thereof. In some embodiments, the at least one functionalization ligand including an aminosilicone group includes at least one primary amine or at least one secondary amine.

In some embodiments, the at least one functionalization ligand including an aminosilicone group includes at least one amine selected from the group consisting of monoamines, diamines, triamines, tetra-amines, penta-amines, hexa-amines, polyamines, and combinations thereof.

In some embodiments, the at least one functionalization ligand including an aminosilicone group includes at least one aminosilicone selected from the group consisting of linear aminosilicones, cyclic aminosilicones, branched aminosilicones, amino-substituted siloxanes, linear amino-substituted disiloxanes, cyclic amino-substituted disiloxanes, linear amino-substituted trisiloxanes, cyclic amino-substituted trisiloxanes, linear amino-substituted tetrasiloxanes, cyclic amino-substituted tetrasiloxanes, linear amino-substituted polysiloxanes, cyclic amino-substituted polysiloxanes, silsesquioxanes, polyoctahedral silsesquioxanes, and combinations thereof.

In some embodiments, the at least one functionalization ligand including an aminosilicone group includes a symmetrical structure. In some embodiments, the at least one functionalization ligand including an aminosilicone group includes an asymmetrical structure.

In some embodiments, when the at least one functionalization ligand including an aminosilicone group includes a disiloxane group, the at least one functionalization ligand including an aminosilicone group includes the same amines on both sides of the disiloxane group. In some embodiments, when the at least one functionalization ligand including an aminosilicone group includes a disiloxane group, the at least one functionalization ligand including an aminosilicone group includes different amines on either side of the disiloxane group.

In some embodiments, the at least one functionalization ligand including an aminosilicone group is an amino-substituted siloxane of Formula (II), Formula (III), Formula (IV), Formula (V), Formula (VI), or Formula (VII)

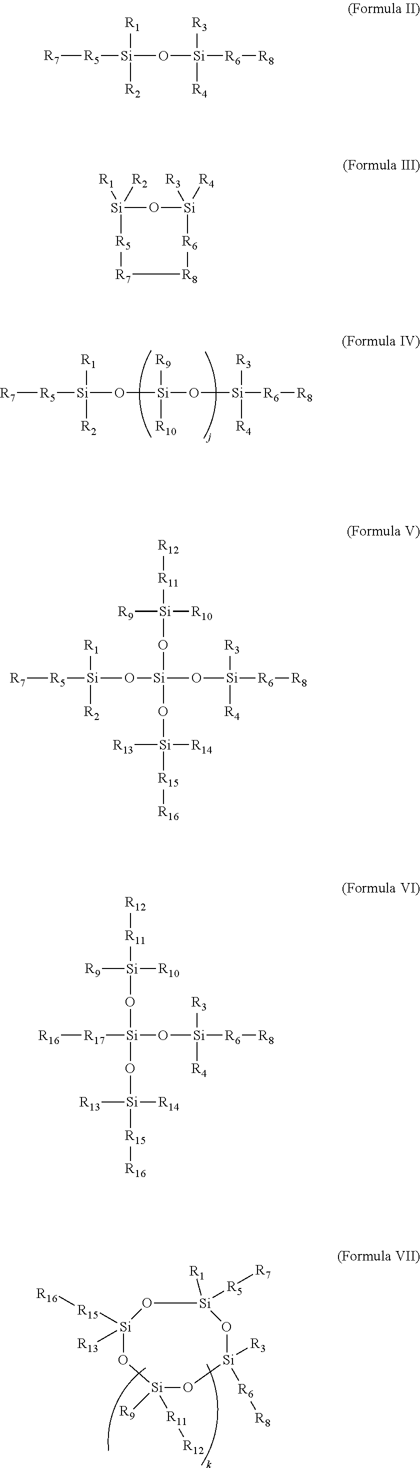

wherein:
R₁, R₂, R₃, R₄, R₉, R₁₀, R₁₃, R₁₄, and R₁₈ are each individually selected from the group consisting of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, substituted or unsubstituted linear heteroalkyl, substituted or unsubstituted branched heteroalkyl, aryl, phenyl, heteroaryl, methyl, ethyl, propyl, isopropyl, butyl, pentyl, and hexyl;
R₅, R₆, R₁₁, R₁₅, and R₁₇ are each individually selected from the group consisting of direct bonds, substituted or unsubstituted C₁-C₆ linear alkyl, substituted or unsubstituted C₃-C₆ branched alkyl, C₁ alkyl, C₂ alkyl, C₃ alkyl, C₄ alkyl, C₅ alkyl, and C₆ alkyl,
R₇, R₈, R₁₂, and R₁₆ are each individually selected from the group consisting of direct bonds, substituted or unsubstituted C₁-C₆ linear alkyl, substituted or unsubstituted C₃-C₆ branched alkyl, C₁ alkyl, C₂ alkyl, C₃ alkyl, C₄ alkyl, C₅ alkyl, C₆ alkyl, and substituents of Formula (VIII)

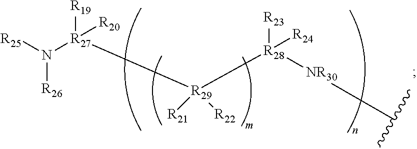

wherein:
the wavy bond indicates a bonding location to Formula (II) or Formula (III) or Formula (IV) or Formula (V) or Formula (VI) or Formula (VII);
R₁₉, R₂₀, R₂₁, R₂₂, R₂₃, and R₂₄ are each individually selected from the group consisting of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, substituted or unsubstituted linear heteroalkyl, substituted or unsubstituted C₁-C₆ linear heteroalkyl, substituted or unsubstituted branched heteroalkyl, substituted or unsubstituted C₃-C₆ branched heteroalkyl, aryl, heteroaryl, methyl, ethyl, propyl, isopropyl, butyl, pentyl, and hexyl;
R₂₅ and R₂₆ are each individually selected from the group consisting of hydrogen, substituted or unsubstituted linear alkyl, substituted or unsubstituted C₁-C₆ linear alkyl, substituted or unsubstituted C₁-C₃ linear alkyl, substituted or unsubstituted branched alkyl, substituted or unsubstituted C₃-C₆ branched alkyl, methyl, ethyl, propyl, substituted or unsubstituted cycloalkyl, substituted or unsubstituted C₃-C₆ cycloalkyl, and substituted or unsubstituted C₄-C₆ cycloalkyl, or, when taken together, R₂₅ and R₂₆ form a single ring selected from the group consisting of heterocycloalkyl and heteroaryl;
R₂₇, R₂₈, and R₂₉ are each individually selected from the group consisting of direct bonds, substituted or unsubstituted C₁-C₆ linear alkyl, substituted or unsubstituted C₃-C₆ branched alkyl, C₁ alkyl, C₂ alkyl, C₃ alkyl, C₄ alkyl, C₅ alkyl, C₆ alkyl, ether, —OCH₂CH₂—, —OCH₂CH₂CH₂—, —OCH₂CH₂CH₂CH₂—, —NHCH₂CH₂—, —NHCH₂CH₂CH₂—, and —NHCH₂CH₂CH₂CH₂—;
R₃₀ is selected from the group consisting of hydrogen, substituted or unsubstituted linear alkyl, substituted or unsubstituted C₁-C₆ linear alkyl, substituted or unsubstituted C₁-C₃ linear alkyl, substituted or unsubstituted branched alkyl, substituted or unsubstituted C₃-C₆ branched alkyl, methyl, ethyl, propyl, substituted or unsubstituted cycloalkyl, substituted or unsubstituted C₃-C₆ cycloalkyl, substituted or unsubstituted C₄-C₆ cycloalkyl, heterocycloalkyl, and heteroaryl;
j is an integer in a range of 0 to 20;
k is an integer in a range of 0 to 20;
m is an integer in a range of 0 to 20; and
n is an integer in a range of 0 to 20.

In some embodiments, the at least one functionalization ligand including an aminosilicone group is selected from the group consisting of

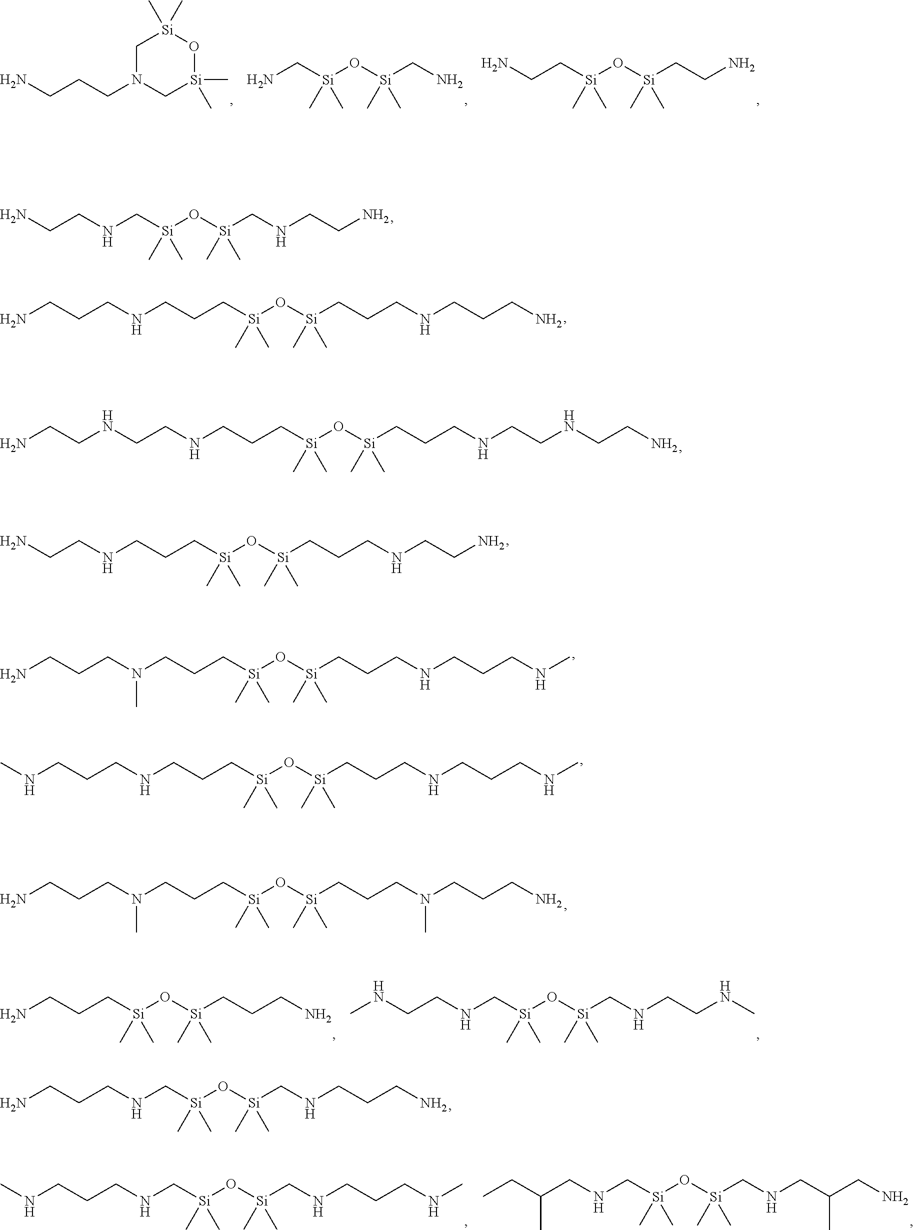

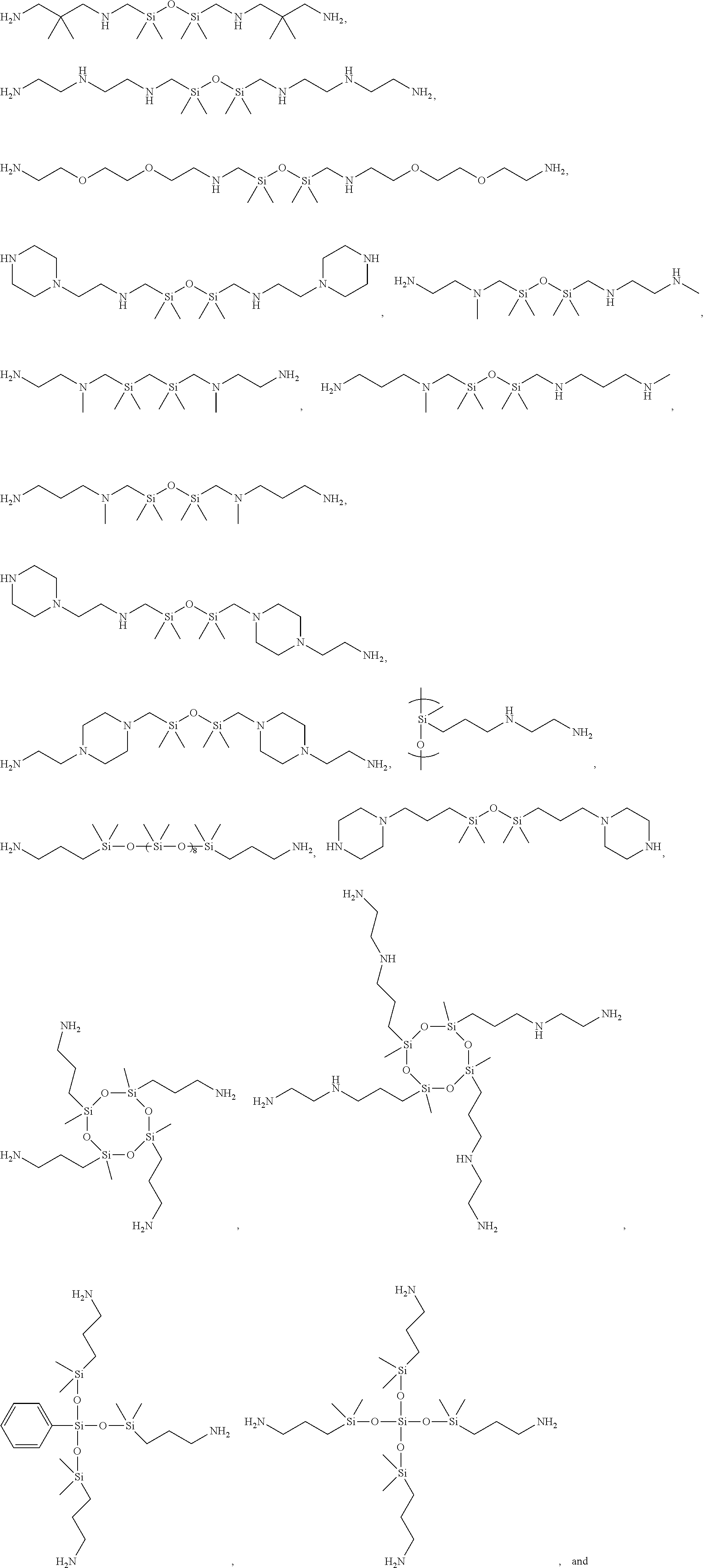

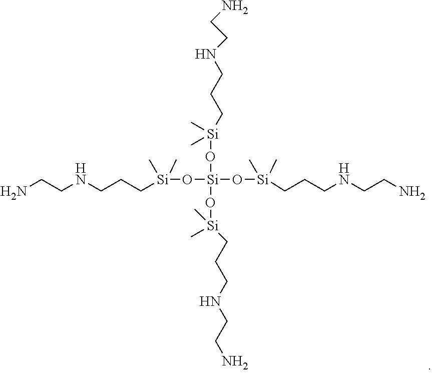 [SMILES: C[Si](C)(CCCNCCN)O[Si](O[Si](C)(C)CCCNCCN)(O[Si](C)(C)CCCNCCN)O[Si](C)(C)CCCNCCN]

FIG. 4 is a schematic of an exemplary control system 400 that may be used to capture CO₂ with a capture system, such as the capture system 100 (shown in FIG. 1) and/or the capture system 200 (shown in FIG. 2). In the exemplary embodiment, the controller 116 includes a memory 402 and a processor 404. The controller 116 may adjust the temperature of one or more adsorption modules 104a-d based on data received by the control system 400 from the contactor sensor 130, such as, but not limited to, the regulated temperature T_{reg} of the first stream 122. The controller 116 may adjust the temperature of one or more adsorption modules 104a-d based on comparisons to data stored in the memory 402, such as desired ranges of the regulated temperature T_{reg}, instructions stored in the memory 402, and/or data analyzed by the processor 404.

Additionally, the controller 116 may adjust the temperature of one or more adsorption modules 104a-d based on data received by the control system 400 from the module sensor 134, such as, but not limited to, the control temperature T_{cntl} and/or the H₂O relative humidity within one or more adsorption modules 104a-d. The controller 116 may adjust the temperature of one or more adsorption modules 104a-d based on comparisons to data stored in the memory 402, such as desired ranges of the control temperature T_{cntl} and/or the H₂O relative humidity, instructions stored in the memory 402, and/or data analyzed by the processor 404.

Exemplary systems for using functionalized chemi-sorbents in the presence of water to facilitate optimizing the adsorption and desorption of carbon dioxide by an adsorbent bed are described herein. The exemplary systems as described herein provide several advantages over conventional designs and processes, including, at least, increasing the efficiency and performance of carbon dioxide adsorption and desorption due to the use of changes in temperature of the adsorbent bed, increasing the efficiency and performance of carbon dioxide adsorption and desorption due to the use of functionalized sorbents within the adsorbent bed, increasing the efficiency and performance of carbon dioxide adsorption and desorption due to the use of changes in water relative humidity within the adsorbent bed, and increasing the performance of the capture system due to the modulation of temperature and water relative humidity within one or more adsorption modules within the adsorbent bed based on the functionalized sorbent within the one or more adsorption modules.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Comparative Moisture Isotherms

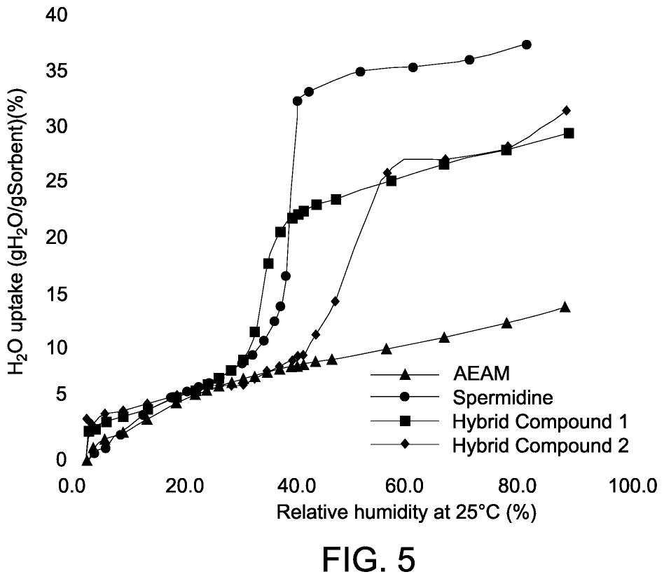

The H₂O adsorption capacity of sorbents functionalized with AEAM, spermidine, a hybrid of spermine: AEAM=0.65:0.35 (“Hybrid Compound 1”), and a hybrid of spermine: AEAM=0.32:0.46 (“Hybrid Compound 2) as a function of relative humidity at 25° C. is shown in FIG. 5. This figure illustrates that the moisture adsorption isotherm, and therefore the relative humidity at which the knee point occurs for monolayer adsorption of H₂O, varies with changes to the chemical structure of the functionalized sorbent. As shown in FIG. 5, all sorbents except AEAM have a moisture adsorption isotherm shape that most closely resembles type IV, including a flattened region leading up to the knee point corresponding to monolayer coverage and adsorption of H₂O at approximately 30% relative humidity for the two hybrid compounds and approximately 35% for Spermidine.

### Example 2. Moisture Adsorption Isotherms of Hybrid Compound 1

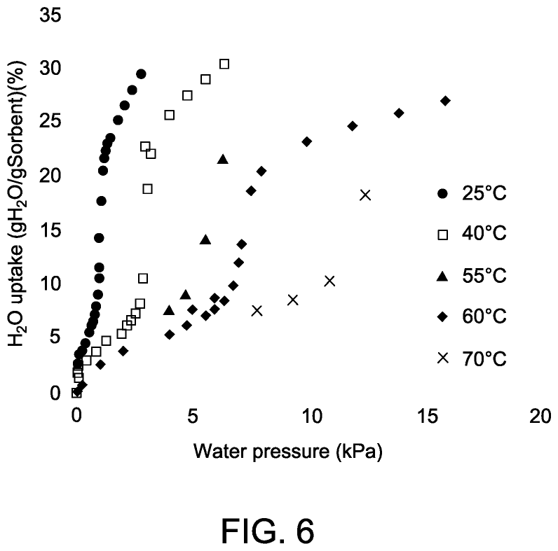

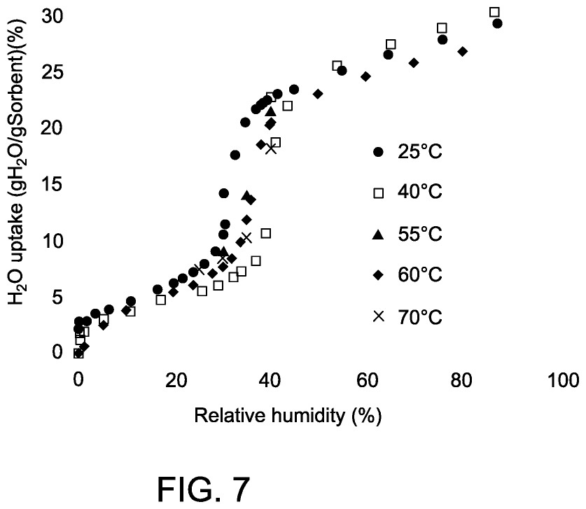

The H₂O adsorption capacity of Hybrid Compound 1 (spermine: AEAM=0.65:0.35) as a function of relative humidity at varying temperatures is shown in FIG. 6. The H₂O adsorption capacity of Hybrid Compound 1 (spermine: AEAM=0.65:0.35) as a function of H₂O partial pressure (kPa) at varying temperatures is shown in FIG. 7. These figures illustrate that although the absolute H2O partial pressure varies, the shape of the moisture isotherms, and therefore the relative humidity at which the knee point occurs for monolayer adsorption of H₂O, stay approximately the same when the temperature changes.

### Example 3. Adsorption Performance of Hybrid Compound 1

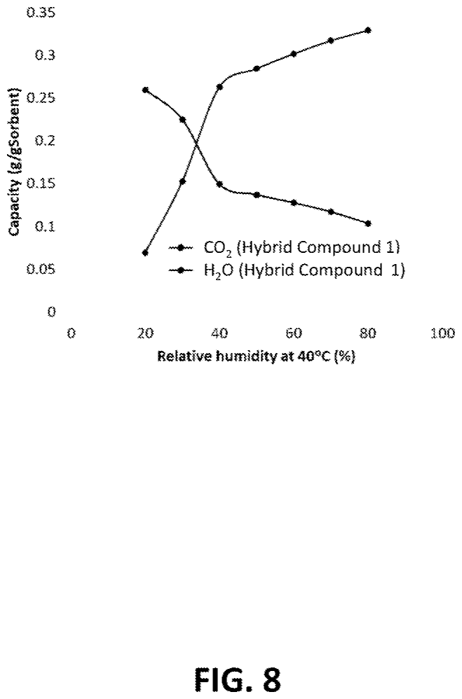

The CO₂ and H₂O adsorption performance of Hybrid Compound 1 (spermine: AEAM=0.65:0.35) as a function of relative humidity at 40° C. is shown in FIG. 8. This figure illustrates that after reaching approximately 30% relative humidity, the adsorption of CO₂ decreases and the adsorption of H₂O increases, in alignment with the knee point for monolayer adsorption of H₂O as shown for Hybrid Compound 1 in FIG. 5.

### Example 4. Adsorption Performances of Hybrid Compound 1 as a Function of Temperature, Water Content and Relative Humidity

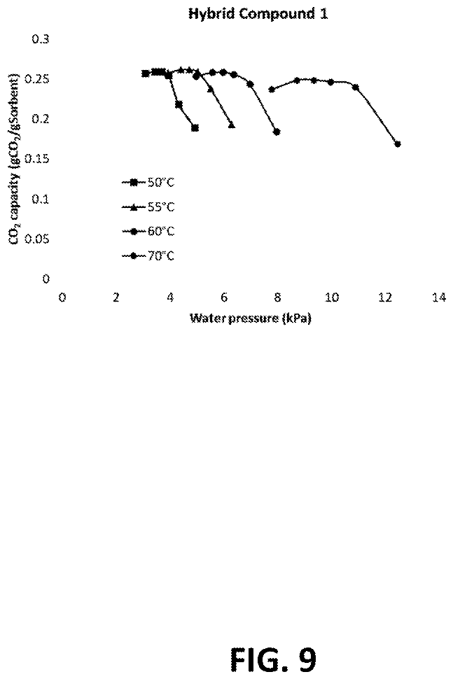

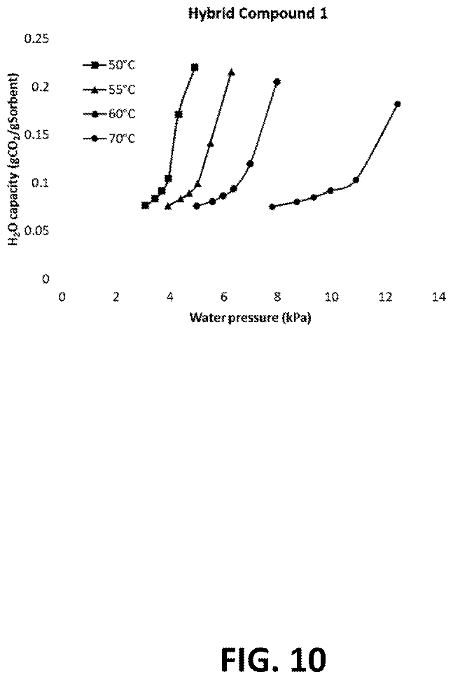

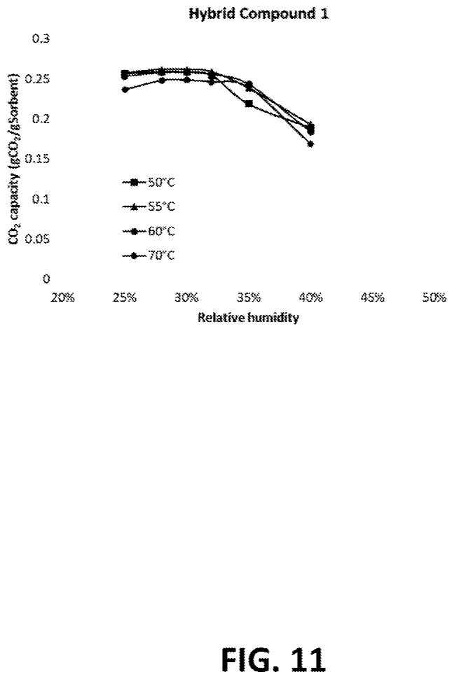

The CO₂ adsorption performance of Hybrid Compound 1 (spermine: AEAM=0.65:0.35) as a function of water pressure (kPa) at varying temperatures is shown in FIG. 9. The H₂O adsorption capacity of Hybrid Compound 1 (spermine: AEAM=0.65:0.35) as a function of water pressure (kPa) at varying temperatures is shown in FIG. 10. The CO₂ adsorption performance of Hybrid Compound 1 (spermine: AEAM=0.65:0.35) as a function of relative humidity at varying temperatures is shown in FIG. 11. These figures illustrate that although the adsorption performance of Hybrid Compound 1 varies in relation to temperature and water pressure, the high performance can be maintained by modulating relative humidity through adjusting temperature and water content.

### Example 5. Comparative Adsorption Capacities of Hybrid Compound 1

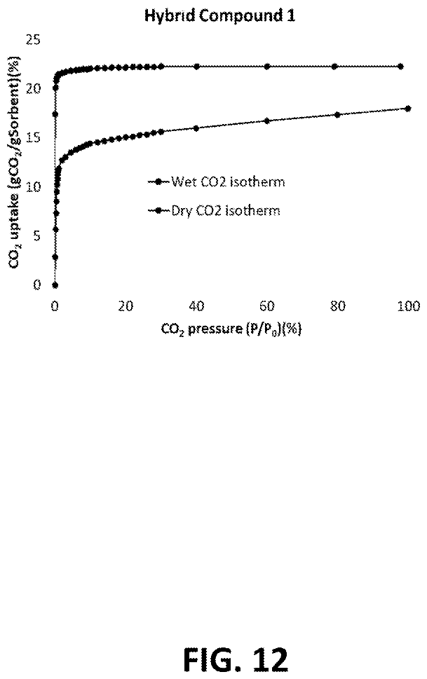

The CO₂ adsorption isotherms of Hybrid Compound 1 (spermine: AEAM=0.65:0.35) for dry CO₂ and wet CO₂ with 30% relative humidity at 40° C. is shown in FIG. 12. This figure illustrates that the adsorption capacity of Hybrid Compound 1 is increased under wet conditions, at 30% relative humidity, as compared to dry conditions.

Exemplary systems for using functionalized chemi-sorbents in the presence of water to facilitate optimizing the adsorption and desorption of carbon dioxide by an adsorbent bed are described herein. The exemplary systems as described herein provide several advantages over conventional designs and processes, including, at least, increasing the efficiency and performance of carbon dioxide adsorption and desorption due to the use of changes in temperature and/or water relative humidity within the adsorbent bed, changes in the functionalized sorbent within the adsorbent bed, and/or increasing the performance of the capture system due to the modulation of temperature and water relative humidity within one or more adsorption modules within the adsorbent bed based on the functionalized sorbent within the one or more adsorption modules.

The above description is meant to be exemplary only, and one skilled in the art will recognize that changes may be made to the embodiments described without departing from the scope of the invention disclosed. Modifications, which fall within the scope of the present invention, will be apparent to those skilled in the art, in light of a review of this disclosure, and such modifications are intended to fall within the appended claims. The systems described herein are not limited to the specific embodiments described herein, but rather portions of the various systems may be utilized independently and separately from other systems described herein.

Although specific features of various embodiments of the invention may be shown in some drawings and not in others, this is for convenience only. Moreover, references to “one embodiment” in the above description are not intended to be interpreted as excluding the existence of additional embodiments that also incorporate the recited features. In accordance with the principles of the invention, any feature of a drawing may be referenced and/or claimed in combination with any feature of any other drawing.

Further aspects of the invention are provided by the subject matter of the following clauses:

A capture system for use in capturing carbon dioxide, the capture system comprising: an adsorbent bed comprising at least one adsorption module and a functionalized sorbent, the at least one adsorption module oriented to: receive a gas stream; adsorb carbon dioxide from the gas stream via the functionalized sorbent; and discharge an exhaust stream; a contactor for use in dynamically controlling a temperature and a relative humidity of the at least one adsorption module; and a controller configured to modulate the temperature and the relative humidity based on the functionalized sorbent to facilitate increasing an amount of carbon dioxide captured by the adsorbent bed.

The capture system in accordance with any of the preceding clauses, wherein the contactor is oriented to receive a regulating fluid for use in dynamically controlling the temperature of the at least one adsorption module.

The capture system in accordance with any of the preceding clauses, further comprising at least one injector oriented to discharge a moisture stream to control a water relative humidity of the at least one adsorption module.

The capture system in accordance with any of the preceding clauses, wherein the controller is further configured to decrease a fluid temperature of the regulating fluid received by the contactor to facilitate increasing the water relative humidity.

The capture system in accordance with any of the preceding clauses, wherein the controller is further configured to modulate the water relative humidity of the at least one adsorption module to facilitate increasing an amount of carbon dioxide captured by the adsorbent bed.

The capture system in accordance with any of the preceding clauses, wherein the controller is further configured to increase the water relative humidity of the at least one adsorption module to facilitate increasing an amount of carbon dioxide adsorbed by the functionalized sorbent.

The capture system in accordance with any of the preceding clauses, wherein the controller is further configured to increase a pressure of the moisture stream discharged from the injector to facilitate increasing the water relative humidity of the at least one adsorption module.

The capture system in accordance with any of the preceding clauses, wherein the controller is further configured to modulate the temperature and the water relative humidity of the at least one adsorption module to achieve a desirable relative humidity based on a moisture adsorption isotherm of the functionalized sorbent.

The capture system in accordance with any of the preceding clauses, wherein the temperature and the water relative humidity of the at least one adsorption module are based on the relative humidity at a knee point of the moisture adsorption isotherm of the functionalized sorbent.

The capture system in accordance with any of the preceding clauses, wherein the at least one adsorption module comprises a plurality of adsorption modules coupled in a series flow arrangement.

The capture system in accordance with any of the preceding clauses, wherein the at least one injector comprises an injector coupled to each of the plurality of adsorption modules.

The capture system in accordance with any of the preceding clauses, wherein the controller is further configured to modulate the temperature and the water relative humidity of each of the plurality of adsorption modules.

The capture system in accordance with any of the preceding clauses, wherein the functionalized sorbent comprises a sorbent and at least one functionalization ligand comprising an amine group.

The capture system in accordance with any of the preceding clauses, wherein the at least one functionalization ligand comprising an amine group comprises at least one amine selected from the group consisting of primary amines, secondary amines, tertiary amines, and combinations thereof.

The capture system in accordance with any of the preceding clauses, wherein the at least one functionalization ligand comprising an amine group comprises at least one amine selected from the group consisting of monoamines, diamines, triamines, tetra-amines, penta-amines, hexa-amines, polyamines, and combinations thereof.

The capture system in accordance with any of the preceding clauses, wherein the functionalized sorbent comprises a sorbent and at least one functionalization ligand comprising an aminosilicone group.

The capture system in accordance with any of the preceding clauses, wherein the functionalized sorbent is a functionalized MOF compound of Formula (I)

$${M}_{x} {L}_{y} {F}_{a}^{A} {F}_{b}^{B} , \qquad ( Formula I )$$

wherein:
M is a MOF metal or metal-containing cluster;
L is a MOF linker;
F^{A} is the at least one functionalization ligand comprising an aminosilicone group;
F^{B} is at least one functionalization ligand not comprising an aminosilicone group;
x is a value in a range of 1 to 6;
y is a value in a range of 1 to 6;
a is a value greater than 0 and less than or equal to 2; and
b is a value in a range of 0 to 2.

The capture system in accordance with any of the preceding clauses, wherein the MOF metal or metal-containing cluster comprises a metal selected from the group consisting of alkali metals, alkaline earth metals, transition metals, Mg, Ca, Mn, Cr, Fe, Co, Ni, Cu, Zn, ions thereof, hydrates thereof, salts thereof, halides thereof, fluorides thereof, chlorides thereof, bromides thereof, iodides thereof, nitrates thereof, acetates thereof, sulfates thereof, phosphates thereof, carbonates thereof, oxides thereof, formates thereof, carboxylates thereof, and combinations thereof.

The capture system in accordance with any of the preceding clauses, wherein the MOF linker comprises a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′: 4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (H₄TDA), 4,4′-Dihydroxyazobenzene-3,3′-dicarboxylic acid (H₄OSA), dicarboxylates, terephthalic acid, tricarboxylates, 1,3,5-benzentricarboxylic acid, azolates, tetrazolates, 1,4-butanedicarboxylic acid, 4-oxopyran-2,6-dicarboxylic acid, 1,6-hexanedicarboxylic acid, decanedicarboxylic acid, 1,8-heptadecanedicarboxylic acid, 1,9-heptadecanedicarboxylic acid, heptadecanedicarboxylic acid, acetylenedicarboxylic acid, 1,2-benzenedicarboxylic acid, 2,3-pyridinedicarboxylic acid, pyridine-2,3-dicarboxylic acid, 1,3-butadiene-1,4-dicarboxylic acid, 1,4-benzenedicarboxylic acid, p-benzenedicarboxylic acid, imidazole-2,4-dicarboxylic acid, 2-methylquinoline-3,4-dicarboxylic acid, quinoline-2,4-dicarboxylic acid, quinoxaline-2,3-dicarboxylic acid, 6-chloroquinoxaline-2,3-dicarboxylic acid, 4,4′-diaminophenylmethane-3,3′-dicarboxylic acid, quinoline-3,4-dicarboxylic acid, 7-chloro-4-hydroxyquinoline-2,8-dicarboxylic acid, diimidedicarboxylic acid, pyridine-2,6-dicarboxylic acid, 2-methylimidazole-4,5-dicarboxylic acid, thiophene-3,4-dicarboxylic acid, 2-isopropylimidazole-4,5-dicarboxylic acid, tetrahydropyran-4,4-dicarboxylic acid, perylene-3,9-dicarboxylic acid, perylenedicarboxylic acid, Pluriol E 200-dicarboxylic acid, 3,6-dioxaoctanedicarboxylic acid, 3,5-cyclohexadiene-1,2-dicarboxylic acid, octanedicarboxylic acid, pentane-3,3-carboxylic acid, 4,4′-diamino-1,1′-diphenyl-3,3′-dicarboxylic acid, 4,4′-diaminodiphenyl-3,3′-dicarboxylic acid, benzidine-3,3′-dicarboxylic acid, 1,4-bis-(phenylamino)benzene-2,5-dicarboxylic acid, 1,1′-dinaphthyl-8,8′-dicarboxylic acid, 7-chloro-8-methylquinoline-2,3-dicarboxylic acid, 1-anilinoanthraquinone-2,4′-dicarboxylic acid, polytetrahydrofuran-250-dicarboxylic acid, 1,4-bis(carboxymethyl) piperazine-2,3-dicarboxylic acid, 7-chloroquinoline-3,8-dicarboxylic acid, 1-(4-carboxy)phenyl-3-(4-chloro) phenylpyrazoline-4,5-dicarboxylic acid, 1,4,5,6,7,7,-hexachloro-5-norbornene-2,3-dicarboxylic acid, phenylindanedicarboxylic acid, 1,3-dibenzyl-2-oxoimidazolidine-4,5-dicarboxylic acid, 1,4-cyclohexanedicarboxylic acid, naphthalene-1,8-dicarboxylic acid, 2-benzoylbenzene-1,3-dicarboxylic acid, 1,3-dibenzyl-2-oxoimidazolidine-4,5-cisdicarboxylic acid, 2,2′-biquinoline-4,4′-dicarboxylic acid, pyridine-3,4-dicarboxylic acid, 3,6,9-trioxaundecanedicarboxylic acid, O-hydroxybenzophenonedicarboxylic acid, Pluriol E 300-dicarboxylic acid, Pluriol E 400-dicarboxylic acid, Pluriol E 600-dicarboxylic acid, pyrazole-3,4-dicarboxylic acid, 2,3-pyrazinedicarboxylic acid, 5,6-dimethyl-2,3-pyrazinedicarboxylic acid, 4,4′-diaminodiphenyletherdiimidedicarboxylic acid, 4,4′-diaminodiphenylmethanediimidedicarboxylic acid, 4,4′-diaminodiphenylsulfonediimidedicarboxylic acid, 2,6-naphthalenedicarboxylic acid, 1,3-adamantanedicarboxylic acid, 1,8-naphthalenedicarboxylic acid, 2,3-naphthalenedicarboxylic acid, 8-methoxy-2,3-naphthalenedicarboxylic acid, 8-nitro-2,3-naphthalenedicarboxylic acid, 8-sulfo-2,3-naphthalenedicarboxylic acid, anthracene-2,3-dicarboxylic acid, 2′-3′-diphenyl-p-terphenyl-4,4″-dicarboxylic acid, diphenylether-4,4′-dicarboxylic acid, imidazole-4,5-dicarboxylic acid, 4 (1H)-oxothiochromene-2,8-dicarboxylic acid, 5-t-butyl-1,3-benzenedicarboxylic acid, 7,8-quinolinedicarboxylic acid, 4,5-imidazoledicarboxylic acid, 4-cyclohexene-1,2-dicarboxylic acid, hexatriacontanedicarboxylic acid, tetradecanedicarboxylic acid, 1,7-heptanedicarboxylic acid, 5-hydroxy-1,3-benzenedicarboxylic acid, pyrazine-2,3-dicarboxylic acid, furan-2,5-dicarboxylic acid, 1-nonene-6,9-dicarboxylic acid, eicosenedicarboxylic acid, 4,4′-dihydroxydiphenylmethane-3,3′-dicarboxylic acid, 1-amino-4-methyl-9,10-dioxo-9,10-dihydroanthracene-2,3-dicarboxylic acid, 2,5-pyridinedicarboxylic acid, cyclohexene-2,3-dicarboxylic acid, 2,9-dichlorofluorubin-4,11-dicarboxylic acid, 7-chloro-3-methylquinoline-6,8-dicarboxylic acid, 2,4-dichlorobenzophenone-2′,5′-dicarboxylic acid, 1,3-benzenedicarboxylic acid, 2,6-pyridinedicarboxylic acid, 1-methylpyrrole-3,4-dicarboxylic acid, 1-benzyl-1H-pyrrole-3,4-dicarboxylic anthraquinone-1,5-dicarboxylic acid, 3,5-pyrazoledicarboxylic acid, 2-nitrobenzene-1,4-dicarboxylic acid, heptane-1,7-dicarboxylic acid, cyclobutane-1,1-dicarboxylic acid, 1,14-tetradecanedicarboxylic acid, 5,6-dehydronorbornane-2,3-dicarboxylic acid, 5-ethyl-2,3-pyridinedicarboxylic acid, 2-hydroxy-1,2,3-propanetricarboxylic acid, 7-chloro-2,3,8-quinolinetricarboxylic acid, 1,2,4-benzenetricarboxylic acid, 1,2,4-butanetricarboxylic acid, 2-phosphono-1,2,4-butanetricarboxylic acid, 1,3,5-benzenetricarboxylic acid, 1-hydroxy-1,2,3-propanetricarboxylic acid, 4,5-dihydro-4,5-dioxo-1H-pyrrolo[2,3-F]quinoline-2,7,9-tricarboxylic acid, 5-acetyl-3-amino-6-methylbenzene-1,2,4-tricarboxylic acid, 3-amino-5-benzoyl-6-methylbenzene-1,2,4-tricarboxylic acid, 1,2,3-propanetricarboxylic acid, aurinetricarboxylic acid, 1,1-dioxide-perylo[1,12-BCD]thiophene-3,4,9,10-tetracarboxylic acid, perylenetetracarboxylic acids, perylene-3,4,9,10-tetracarboxylic acid, perylene-1,12-sulfone-3,4,9,10-tetracarboxylic acid, butanetetracarboxylic acids, 1,2,3,4-butanetetracarboxylic acid, meso-1,2,3,4-butanetetracarboxylic acid, decane-2,4,6,8-tetracarboxylic acid, 1,4,7,10,13,16-hexaoxacyclooctadecane-2,3,11,12-tetracarboxylic acid, 1,2,4,5-benzenetetracarboxylic acid, 1,2,11,12-dodecanetetracarboxylic acid, 1,2,5,6-hexanetetracarboxylic acid, 1,2,7,8-octanetetracarboxylic acid, 1,4,5,8-naphthalenetetracarboxylic acid, 1,2,9,10-decanetetracarboxylic acid, benzophenonetetracarboxylic acid, 3,3′,4,4′-benzophenonetetracarboxylic acid, tetrahydrofurantetracarboxylic acid, cyclopentanetetracarboxylic acids, cyclopentane-1,2,3,4-tetracarboxylic acid, polytopic linkers, ditopic linkers, tritopic linkers, tetratopic linkers, pentatopic linkers, hexatopic linkers, heptatopic linkers, octatopic linkers, mixed linkers, desymmetrized linker, metallo linkers, N-heterocyclic linkers, protonated, partially and fully deprotonated forms thereof, and combinations thereof.

The capture system in accordance with any of the preceding clauses, wherein the at least one functionalization ligand comprising an aminosilicone group comprises at least one aminosilicone selected from the group consisting of linear aminosilicones, cyclic aminosilicones, branched aminosilicones, amino-substituted siloxanes, linear amino-substituted disiloxanes, cyclic amino-substituted disiloxanes, linear amino-substituted trisiloxanes, cyclic amino-substituted trisiloxanes, linear amino-substituted tetrasiloxanes, cyclic amino-substituted tetrasiloxanes, linear amino-substituted polysiloxanes, cyclic amino-substituted polysiloxanes, silsesquioxanes, polyoctahedral silsesquioxanes, and combinations thereof.

While the invention has been described in terms of various specific embodiments, those skilled in the art will recognize that the invention can be practiced with modification within the spirit and scope of the claims.

What is claimed is:

1. A capture system for use in capturing carbon dioxide, the capture system comprising:
an adsorbent bed comprising at least one adsorption module and a functionalized sorbent, the at least one adsorption module oriented to:
receive a gas stream;
adsorb carbon dioxide from the gas stream via the functionalized sorbent; and
discharge an exhaust stream;
a contactor for use in dynamically controlling a temperature and a relative humidity of the at least one adsorption module; and
a controller configured to modulate the temperature and the relative humidity based on the functionalized sorbent to facilitate increasing an amount of carbon dioxide captured by the adsorbent bed.

2. The capture system in accordance with claim 1, wherein the contactor is oriented to receive a regulating fluid for use in dynamically controlling the temperature of the at least one adsorption module.

3. The capture system in accordance with claim 2, further comprising at least one injector oriented to discharge a moisture stream to control a water relative humidity of the at least one adsorption module.

4. The capture system in accordance with claim 3, wherein the controller is further configured to decrease a fluid temperature of the regulating fluid received by the contactor to facilitate increasing the water relative humidity.

5. The capture system in accordance with claim 4, wherein the controller is further configured to modulate the water relative humidity of the at least one adsorption module to facilitate increasing an amount of carbon dioxide captured by the adsorbent bed.

6. The capture system in accordance with claim 5, wherein the controller is further configured to increase the water relative humidity of the at least one adsorption module to facilitate increasing an amount of carbon dioxide adsorbed by the functionalized sorbent.

7. The capture system in accordance with claim 5, wherein the controller is further configured to increase a pressure of the moisture stream discharged from the injector to facilitate increasing the water relative humidity of the at least one adsorption module.

8. The capture system in accordance with claim 5, wherein the controller is further configured to modulate the temperature and the water relative humidity of the at least one adsorption module to reach a desirable relative humidity based on a moisture adsorption isotherm of the functionalized sorbent.

9. The capture system in accordance with claim 8, wherein the temperature and the water relative humidity of the at least one adsorption module are based on the relative humidity at a knee point of the moisture adsorption isotherm of the functionalized sorbent.

10. The capture system in accordance with claim 4, wherein the at least one adsorption module comprises a plurality of adsorption modules coupled in a series flow arrangement.

11. The capture system in accordance with claim 10, wherein the at least one injector comprises an injector coupled to each of the plurality of adsorption modules.

12. The capture system in accordance with claim 11, wherein the controller is further configured to modulate the temperature and the water relative humidity of each of the plurality of adsorption modules.

13. The capture system in accordance with claim 1, wherein the functionalized sorbent comprises a sorbent and at least one functionalization ligand comprising an amine group.

14. The capture system in accordance with claim 13, wherein the at least one functionalization ligand comprising an amine group comprises at least one amine selected from the group consisting of primary amines, secondary amines, tertiary amines, and combinations thereof.

15. The capture system in accordance with claim 13, wherein the at least one functionalization ligand comprising an amine group comprises at least one amine selected from the group consisting of monoamines, diamines, triamines, tetra-amines, penta-amines, hexa-amines, polyamines, and combinations thereof.

16. The capture system in accordance with claim 1, wherein the functionalized sorbent comprises a sorbent and at least one functionalization ligand comprising an aminosilicone group.

17. The capture system in accordance with claim 16, wherein the functionalized sorbent is a functionalized MOF compound of Formula (I)
$${M}_{x} {L}_{y} {F}_{a}^{A} {F}_{b}^{B} , \qquad ( Formula I )$$
wherein:
M is a MOF metal or metal-containing cluster;
L is a MOF linker;
F^{A} is the at least one functionalization ligand comprising an aminosilicone group;
F^{B} is at least one functionalization ligand not comprising an aminosilicone group;
x is a value in a range of 1 to 6;
y is a value in a range of 1 to 6;
a is a value greater than 0 and less than or equal to 2; and
b is a value in a range of 0 to 2.

18. The capture system in accordance with claim 17, wherein the MOF metal or metal-containing cluster comprises a metal selected from the group consisting of alkali metals, alkaline earth metals, transition metals, Mg, Ca, Mn, Cr, Fe, Co, Ni, Cu, Zn, ions thereof, hydrates thereof, salts thereof, halides thereof, fluorides thereof, chlorides thereof, bromides thereof, iodides thereof, nitrates thereof, acetates thereof, sulfates thereof, phosphates thereof, carbonates thereof, oxides thereof, formates thereof, carboxylates thereof, and combinations thereof.

19. The capture system in accordance with claim 17, wherein the MOF linker comprises a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′: 4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (HATDA), 4,4′-Dihydroxyazobenzene-3,3′-dicarboxylic acid (H₄OSA), dicarboxylates, terephthalic acid, tricarboxylates, 1,3,5-benzentricarboxylic acid, azolates, tetrazolates, 1,4-butanedicarboxylic acid, 4-oxopyran-2,6-dicarboxylic acid, 1,6-hexanedicarboxylic acid, decanedicarboxylic acid, 1,8-heptadecanedicarboxylic acid, 1,9-heptadecanedicarboxylic acid, heptadecanedicarboxylic acid, acetylenedicarboxylic acid, 1,2-benzenedicarboxylic acid, 2,3-pyridinedicarboxylic acid, pyridine-2,3-dicarboxylic acid, 1,3-butadiene-1,4-dicarboxylic acid, 1,4-benzenedicarboxylic acid, p-benzenedicarboxylic acid, imidazole-2,4-dicarboxylic acid, 2-methylquinoline-3,4-dicarboxylic acid, quinoline-2,4-dicarboxylic acid, quinoxaline-2,3-dicarboxylic acid, 6-chloroquinoxaline-2,3-dicarboxylic acid, 4,4′-diaminophenylmethane-3,3′-dicarboxylic acid, quinoline-3,4-dicarboxylic acid, 7-chloro-4-hydroxyquinoline-2,8-dicarboxylic acid, diimidedicarboxylic acid, pyridine-2,6-dicarboxylic acid, 2-methylimidazole-4,5-dicarboxylic acid, thiophene-3,4-dicarboxylic acid, 2-isopropylimidazole-4,5-dicarboxylic acid, tetrahydropyran-4,4-dicarboxylic acid, perylene-3,9-dicarboxylic acid, perylenedicarboxylic acid, Pluriol E 200-dicarboxylic acid, 3,6-dioxaoctanedicarboxylic acid, 3,5-cyclohexadiene-1,2-dicarboxylic acid, octanedicarboxylic acid, pentane-3,3-carboxylic acid, 4,4′-diamino-1,1′-diphenyl-3,3′-dicarboxylic acid, 4,4′-diaminodiphenyl-3,3′-dicarboxylic acid, benzidine-3,3′-dicarboxylic acid, 1,4-bis-(phenylamino)benzene-2,5-dicarboxylic acid, 1,1′-dinaphthyl-8,8′-dicarboxylic acid, 7-chloro-8-methylquinoline-2,3-dicarboxylic acid, 1-anilinoanthraquinone-2,4′-dicarboxylic acid, polytetrahydrofuran-250-dicarboxylic acid, 1,4-bis(carboxymethyl) piperazine-2,3-dicarboxylic acid, 7-chloroquinoline-3,8-dicarboxylic acid, 1-(4-carboxy)phenyl-3-(4-chloro) phenylpyrazoline-4,5-dicarboxylic acid, 1,4,5,6,7,7,-hexachloro-5-norbornene-2,3-dicarboxylic acid, phenylindanedicarboxylic acid, 1,3-dibenzyl-2-oxoimidazolidine-4,5-dicarboxylic acid, 1,4-cyclohexanedicarboxylic acid, naphthalene-1,8-dicarboxylic acid, 2-benzoylbenzene-1,3-dicarboxylic acid, 1,3-dibenzyl-2-oxoimidazolidine-4,5-cisdicarboxylic acid, 2,2′-biquinoline-4,4′-dicarboxylic acid, pyridine-3,4-dicarboxylic acid, 3,6,9-trioxaundecanedicarboxylic acid, o-hydroxybenzophenonedicarboxylic acid, Pluriol E 300-dicarboxylic acid, Pluriol E 400-dicarboxylic acid, Pluriol E 600-dicarboxylic acid, pyrazole-3,4-dicarboxylic acid, 2,3-pyrazinedicarboxylic acid, 5,6-dimethyl-2,3-pyrazinedicarboxylic acid, 4,4′-diaminodiphenyletherdiimidedicarboxylic acid, 4,4′-diaminodiphenylmethanediimidedicarboxylic acid, 4,4′-diaminodiphenylsulfonediimidedicarboxylic acid, 2,6-naphthalenedicarboxylic acid, 1,3-adamantanedicarboxylic acid, 1,8-naphthalenedicarboxylic acid, 2,3-naphthalenedicarboxylic acid, 8-methoxy-2,3-naphthalenedicarboxylic acid, 8-nitro-2,3-naphthalenedicarboxylic acid, 8-sulfo-2,3-naphthalenedicarboxylic acid, anthracene-2,3-dicarboxylic acid, 2′-3′-diphenyl-p-terphenyl-4,4″-dicarboxylic acid, diphenylether-4,4′-dicarboxylic acid, imidazole-4,5-dicarboxylic acid, 4 (1H)-oxothiochromene-2,8-dicarboxylic acid, 5-t-butyl-1,3-benzenedicarboxylic acid, 7,8-quinolinedicarboxylic acid, 4,5-imidazoledicarboxylic acid, 4-cyclohexene-1,2-dicarboxylic acid, hexatriacontanedicarboxylic acid, tetradecanedicarboxylic acid, 1,7-heptanedicarboxylic acid, 5-hydroxy-1,3-benzenedicarboxylic acid, pyrazine-2,3-dicarboxylic acid, furan-2,5-dicarboxylic acid, 1-nonene-6,9-dicarboxylic acid, eicosenedicarboxylic acid, 4,4′-dihydroxydiphenylmethane-3,3′-dicarboxylic acid, 1-amino-4-methyl-9,10-dioxo-9,10-dihydroanthracene-2,3-dicarboxylic acid, 2,5-pyridinedicarboxylic acid, cyclohexene-2,3-dicarboxylic acid, 2,9-dichlorofluorubin-4,11-dicarboxylic acid, 7-chloro-3-methylquinoline-6,8-dicarboxylic acid, 2,4-dichlorobenzophenone-2′,5′-dicarboxylic acid, 1,3-benzenedicarboxylic acid, 2,6-pyridinedicarboxylic acid, 1-methylpyrrole-3,4-dicarboxylic acid, 1-benzyl-1H-pyrrole-3,4-dicarboxylic acid, anthraquinone-1,5-dicarboxylic acid, 3,5-pyrazoledicarboxylic acid, 2-nitrobenzene-1,4-dicarboxylic acid, heptane-1,7-dicarboxylic acid, cyclobutane-1,1-dicarboxylic acid, 1,14-tetradecanedicarboxylic acid, 5,6-dehydronorbornane-2,3-dicarboxylic acid, 5-ethyl-2,3-pyridinedicarboxylic acid, 2-hydroxy-1,2,3-propanetricarboxylic acid, 7-chloro-2,3,8-quinolinetricarboxylic acid, 1,2,4-benzenetricarboxylic acid, 1,2,4-butanetricarboxylic acid, 2-phosphono-1,2,4-butanetricarboxylic acid, 1,3,5-benzenetricarboxylic acid, 1-hydroxy-1,2,3-propanetricarboxylic acid, 4,5-dihydro-4,5-dioxo-1H-pyrrolo[2,3-F]quinoline-2,7,9-tricarboxylic acid, 5-acetyl-3-amino-6-methylbenzene-1,2,4-tricarboxylic acid, 3-amino-5-benzoyl-6-methylbenzene-1,2,4-tricarboxylic acid, 1,2,3-propanetricarboxylic acid, aurinetricarboxylic acid, 1,1-dioxide-perylo[1,12-BCD]thiophene-3,4,9,10-tetracarboxylic acid, perylenetetracarboxylic acids, perylene-3,4,9,10-tetracarboxylic acid, perylene-1,12-sulfone-3,4,9,10-tetracarboxylic acid, butanetetracarboxylic acids, 1,2,3,4-butanetetracarboxylic acid, meso-1,2,3,4-butanetetracarboxylic acid, decane-2,4,6,8-tetracarboxylic acid, 1,4,7,10,13,16-hexaoxacyclooctadecane-2,3,11,12-tetracarboxylic acid, 1,2,4,5-benzenetetracarboxylic acid, 1,2,11,12-dodecanetetracarboxylic acid, 1,2,5,6-hexanetetracarboxylic acid, 1,2,7,8-octanetetracarboxylic acid, 1,4,5,8-naphthalenetetracarboxylic acid, 1,2,9,10-decanetetracarboxylic acid, benzophenonetetracarboxylic acid, 3,3′,4,4′-benzophenonetetracarboxylic acid, tetrahydrofurantetracarboxylic acid, cyclopentanetetracarboxylic acids, cyclopentane-1,2,3,4-tetracarboxylic acid, polytopic linkers, ditopic linkers, tritopic linkers, tetratopic linkers, pentatopic linkers, hexatopic linkers, heptatopic linkers, octatopic linkers, mixed linkers, desymmetrized linker, metallo linkers, N-heterocyclic linkers, protonated, partially and fully deprotonated forms thereof, and combinations thereof.

20. The capture system in accordance with claim 17, wherein the at least one functionalization ligand comprising an aminosilicone group comprises at least one aminosilicone selected from the group consisting of linear aminosilicones, cyclic aminosilicones, branched aminosilicones, amino-substituted siloxanes, linear amino-substituted disiloxanes, cyclic amino-substituted disiloxanes, linear amino-substituted trisiloxanes, cyclic amino-substituted trisiloxanes, linear amino-substituted tetrasiloxanes, cyclic amino-substituted tetrasiloxanes, linear amino-substituted polysiloxanes, cyclic amino-substituted polysiloxanes, silsesquioxanes, polyoctahedral silsesquioxanes, and combinations thereof.
